(19) United States
(12) Patent Application Publication
(10) Pub. No.: US 2026/0337601 A1
(43) Pub. Date: Oct. 29, 2026

(54) SYSTEMS AND METHODS FOR SCREENING OF LARGE GENE LIBRARIES

(71) Applicant: President and Fellows of Harvard College, Cambridge, MA (US)

(72) Inventors: David A. Weitz, Cambridge, MA (US); Karla Milcic, Cambridge, MA (US); Xinge Zhang, Cambridge, MA (US); Anqi Chen, Cambridge, MA (US)

(73) Assignee: President and Fellows of Harvard College, Cambridge, MA (US)

(21) Appl. No.: 19/115,049

(22) PCT Filed: Sep. 25, 2023
(86) PCT No.: PCT/US2023/075065

§ 371 Date: Mar. 25, 2025

Related U.S. Application Data
(60) Provisional application No. 63/410,116, filed on Sep. 26, 2022.
(60) Provisional application No. 63/410,140, filed on Sep. 26, 2022.

(51) Int. Cl. C12N 15/10

(52) U.S. Cl. CPC C12N 15/1068; C12N 15/1031

(57) ABSTRACT

Library screening is an important analytic tool for identifying functional nucleic acids or proteins. In some aspects, droplet-based systems and methods for library screening are provided. The systems and methods may include steps of sorting droplets based on activity, amplifying nucleic acids of “activity droplets” having high activity, and separating nucleic acids from the activity droplets into new droplets. The steps may be iterated, such that activity droplets from successive iterations tend to include fewer nucleic acids. Such approaches may facilitate identification of active nucleic acids, or of nucleic acids encoding active proteins.

### RELATED APPLICATIONS

This application claims the benefit of U.S. Provisional Patent Application Ser. No. 63/410,140, filed Sep. 26, 2022, entitled “Systems and Methods for Screening of Large Gene Libraries,” by Weitz, et al., and U.S. Provisional Patent Application Ser. No. 63/410,116, filed Sep. 26, 2022, entitled “Methods and Systems for Full Gene Length Single Point Mutagenesis,” by Weitz, et al. Each of these is incorporated herein by reference in its entirety.

### TECHNICAL FIELD

Methods and associated systems or articles for screening of large gene libraries are generally described.

### BACKGROUND

A library of molecules may be screened based on activity of a library molecule, or on activity of an associated molecule, such as a protein expressed by the library molecule. Conventional methods for library screening limit a number of variants that may be screened. Screening for protein expression (e.g., enzyme expression) by a library can be particularly difficult using conventional methods. Accurate methods of screening larger libraries are desirable.

### SUMMARY

Library screening is an important analytic tool for identifying functional nucleic acids or proteins. In some aspects, droplet-based systems and methods for library screening are provided. The systems and methods may include steps of sorting droplets based on activity, amplifying nucleic acids of “activity droplets” having high activity, and separating nucleic acids from the activity droplets into new droplets. The steps may be iterated, such that activity droplets from successive iterations tend to include fewer nucleic acids. Such approaches may facilitate identification of active nucleic acids, or of nucleic acids encoding active proteins. The subject matter of the present disclosure involves, in some cases, interrelated products, alternative solutions to a particular problem, and/or a plurality of different uses of one or more systems and/or articles.

According to one aspect, a method is provided. According to some embodiments, the method, comprises: determining one or more activity droplets of a first plurality of droplets having an activity of a target substrate, wherein at least 50% of the droplets the first plurality of droplets each comprise at least 5 distinct nucleic acid sequences; separating nucleic acids from the one or more activity droplets of the first plurality of droplets into a second plurality of droplets; and amplifying the nucleic acids of the one or more activity droplets.

According to another aspect, a method is provided. According to some embodiments, the method comprises: determining one or more activity droplets of a first plurality of droplets having an activity of a target substrate, wherein the first plurality of droplets comprises greater than or equal to 10⁵ droplets containing the target substrate, and wherein at least 50% of the droplets the first plurality of droplets each comprise at least 10⁵ distinct nucleic acid sequences; separating nucleic acids from the one or more activity droplets of the first plurality of droplets into a second plurality of droplets; and amplifying the nucleic acids of the one or more activity droplets.

In another aspect, a method is provided. According to some embodiments, the method comprises: in a first plurality of droplets comprising nucleic acids, translating proteins from the nucleic acids within the droplets, at least 50% of the droplets the first plurality of droplets containing therein at least 10⁵ distinct nucleic acid sequences, and wherein the first plurality of droplets comprises greater than or equal to 10⁵ droplets having distinct nucleic acid sequences contained therein; determining one or more activity droplets of the first plurality of droplets that contain activity of a target substrate; separating nucleic acids from the one or more activity droplets of the first plurality of droplets into a second plurality of droplets; and amplifying the nucleic acids of the one or more activity droplets.

In still another aspect, an article is provided. In some embodiments, the article comprises: a plurality of droplets, at least 50% of the droplets containing therein at least 10⁵ distinct nucleic acid sequences and a target substrate, wherein the plurality of droplets comprises greater than or equal to 10⁵ droplets having distinct nucleic acid sequences contained therein.

In another aspect, a composition is provided. According to some embodiments, the composition, comprises: an amino acid sequence at least 70% identical to one of Seq. ID. Nos. 2-24, wherein the amino acid sequence is not Seq. ID. No. 25.

In one aspect, a composition is provided. According to some embodiments, the composition, comprises: a nucleic acid sequence at least 70% identical to one of Seq. ID. Nos. 26-48, wherein the nucleic acid sequence is not Seq. ID. No. 49.

Other advantages and novel features of the present disclosure will become apparent from the following detailed description of various non-limiting embodiments of the disclosure when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present disclosure will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the disclosure shown where illustration is not necessary to allow those of ordinary skill in the art to understand the disclosure. In the figures:

FIG. 1A presents a schematic illustration of a nucleic acid, according to some embodiments;

FIG. 1B presents a schematic illustration of a nucleic acid, according to some embodiments;

FIG. 1C presents a schematic illustration of a nucleic acid, according to some embodiments;

FIG. 2A presents a schematic illustration of a method of screening a large library, according to some embodiments;

FIG. 2B presents a schematic illustration of a method of screening a large library, according to some embodiments;

FIG. 3A presents a schematic flow diagram of a method of screening a large library, according to some embodiments;

FIG. 3B presents a schematic flow diagram of a method of screening a large library, according to some embodiments;

FIG. 3C presents a schematic flow diagram of a method of screening a large library, according to some embodiments;

FIG. 3D presents a schematic flow diagram of a method of screening a large library, according to some embodiments;

FIG. 4 presents a schematic illustration of a circular nucleic acid and a plurality of primers suitable for preparing a nucleic acid library, according to some embodiments;

FIG. 5A presents a schematic illustration of a nucleic acid and pluralities of primers, according to some embodiments;

FIG. 5B presents a schematic illustration of a nucleic acid and pluralities of primers, according to some embodiments;

FIG. 5C presents a schematic illustration of a nucleic acid and pluralities of primers, according to some embodiments;

FIG. 5D presents a schematic illustration of nucleic acids representing libraries of nucleic acids, according to some embodiments;

FIG. 5E presents a schematic illustration of nucleic acids representing a library of nucleic acids, according to some embodiments;

FIG. 6A presents a confocal microscope image of a plurality of droplets including activity droplets, according to some embodiments;

FIG. 6B presents a red fluorescence image of a plurality of droplets including activity droplets, according to some embodiments;

FIG. 6C presents a green fluorescence image of a plurality of droplets including activity droplets, according to some embodiments;

FIGS. 7A-7C present non-limiting schematic illustrations of microfluidic devices, according to some embodiments;

FIG. 8A presents a non-limiting, schematic illustration of cell-free protein synthesis in drops, according to some embodiments;

FIG. 8B presents a non-limiting, schematic illustration of pico-injection of a substrate, followed by fluorescence-based droplet sorting, according to some embodiments;

FIG. 8C presents a non-limiting, schematic illustration of co-encapsulation of one bacteria cell host expressing enzyme mutants, fluorescent substrate, and lysis buffer, followed by fluorescence-based droplet sorting, according to some embodiments;

FIG. 9 presents a non-limiting, schematic illustration of the sorting of activity droplets, according to some embodiments; and

FIG. 10 presents the activity (in arbitrary units) of various sequences after thermal shock at 80 C as a function of the concentration of Tween, according to some embodiments.

### DETAILED DESCRIPTION

Refinement of active, functional molecules from large libraries is an important experimental method that is often constrained by limitations of resolution, detection, and processing methods. Microfluidics offer a number of processing advantages for handling molecular libraries, but detection of dilute molecules in individual droplets can present certain challenges. Conventional cell-based methods of library refinement such as phage display are also limiting, and may restrict the size of a molecular library to a number of molecules on the order of 10⁸. The systems and methods described herein permit library refinement of large molecular libraries (e.g., libraries including a number of molecules on the order of 10¹²) using microfluidic systems. Disclosed herein are systems and methods for partitioning molecular libraries into microfluidic droplets and selecting for droplets based on activation of a target substrate.

To provide a single, nonlimiting example, included solely for illustrative purposes, in some embodiments, a method comprises partitioning a library of ~10¹² DNA sequences into a plurality of ~10⁴ droplets such that each droplet comprises at least 10⁸ distinct DNA sequences. The number of distinct DNA molecules per droplet may be controlled, for example, by controlling the DNA concentration and droplet size such that droplets are statistically likely to include the desired number of DNA sequences. In this way, each droplet may act as an independent library of sequences. In vitro transcription and translation may be performed in the droplets, and a target molecule may be included in the droplets such that droplets produce a signal if they include an active protein encoded by the DNA library, and do not produce a signal if they do not include an active protein encoded by the DNA library.

An iterative process of selecting droplets for activity of the target substrate, amplifying the DNA sequences within the active droplets by PCR, and breaking the active droplets into a new plurality of droplets may be used to further differentiate nucleic acids expressing active proteins from nucleic acids that merely express inactive proteins. By this approach, active DNA strands may be refined from the library, and may be amplified, sequenced, or put to any other functional purpose for which protein activity is desired.

Techniques for screening nucleic acid libraries are known in the prior art. Some common techniques, such as phage display, involve expressing the library in vivo to determine activity. However, such processes can limit the library size to ~10⁸. Other library techniques, such as mRNA display, allow larger libraries, but are limited by sequence size, require the coupling of an expressed protein to its source RNA during translation, and are not typically useful when screening for catalytic activity. Methods of the type disclosed herein may improve library size without imposing burdensome limitations on the composition of the library itself. This is an advantage of the specific method outlined above. Of course, the disclosure is not limited to so specific a method, and a number of other systems and methods are described herein.

The present invention generally relates, in certain aspects, to droplet-based microfluidic devices and methods. It may be useful, in some embodiments, to identify activity of a target substrate within a droplet. In certain aspects, a method comprises determining one or more activity droplets of a plurality of droplets that have an activity of a target substrate. The target substrate may be any of a variety of appropriate target substrates. For example, the target substrate may be a binding target (e.g., a viral or cancer cell antigen), a reaction target (e.g., a molecule that should react with or undergo a reaction catalyzed by a molecule in the droplet), or may be a target configured to activate as a result of any of a variety of other suitable biochemical processes, etc.

Any of a variety of approaches may be used to determine the activity droplets having the activity of the target substrate. For example, some embodiments may comprise identifying activity droplets that include any activity of a target substrate. However, some embodiments are directed to identifying a subset of droplets that includes activity exceeding a threshold value. For example, a target substrate may, upon activation, produce a signal (e.g., a colorimetric, fluorescent, or luminescent signal) exceeding a predefined minimum signal. Such an approach may be useful in certain cases for identifying highly active molecules, e.g., while excluding less active molecules. It should, of course, be understood that the droplets with the most activity do not necessarily correspond to the droplets comprising the most active molecules. For example, some droplets may stochastically include larger numbers of active sequences, thereby demonstrating higher apparent activity without necessarily including highly active variants. More generally, it should be understood that separation of active droplets may be performed by any of a variety of suitable systems and methods, e.g., as described herein, as the disclosure is not so limited.

Some aspects are generally directed to systems and methods of determining one or more nucleic acids in a sample (e.g., determining one or more nucleic acids that can cause activation of a target substrate). In some cases, the nucleic acids may be encapsulated into droplets. In some cases, the nucleic acids are encapsulated at relatively low concentrations, e.g., such that the droplets may, on the average contain less than 1 nucleic acid per droplet. This may be useful to ensure that most or all of the nucleic acids are transcribed, translated, or amplified, e.g., substantially evenly. In contrast, if the nucleic acids were to be transcribed, translated, or amplified in bulk solution, some nucleic acids could be transcribed, translated, or amplified without others being transcribed, translated, or amplified (or merely being transcribed, translated, or amplified to a much lesser degree). Thus, in certain embodiments as described herein the nucleic acids are encapsulated into droplets, and manipulated therein.

In some embodiments, a plurality of droplets may contain greater than or equal to 10⁶, greater than or equal to 10⁷, greater than or equal to 10⁸, greater than or equal to 10⁹, greater than or equal to 10¹⁰, greater than or equal to 10¹¹, greater than or equal to 10¹² or more distinct nucleic acid sequences within the droplets. In some embodiments, a plurality of droplets contains less than or equal to 10¹⁴, less than or equal to 10¹³, less than or equal to 10¹², less than or equal to 10¹¹, less than or equal to 10¹⁰, less than or equal to 10⁹, or less distinct nucleic acid sequences within the droplets. Combinations of these ranges are possible. For example, in some embodiments, a plurality of droplets contains greater than or equal to 10⁶ and less than or equal to 10¹⁴ distinct nucleic acid sequences within the droplets. Other ranges are also possible.

In some embodiments, droplets of the first plurality of droplets contain greater than or equal to 5, greater than or equal to 10, greater than or equal to 10², greater than or equal to 10³, greater than or equal to 10⁴, greater than or equal to 10⁵, greater than or equal to 10⁶, greater than or equal to 10⁷, or more distinct nucleic acid sequences. In some embodiments, droplets of the first plurality of droplets contain less than or equal to 10⁸, less than or equal to 10⁷, less than or equal to 10⁶, less than or equal to 10⁵, or less distinct nucleic acid sequences. Combinations of these ranges are possible. For example, in some embodiments, droplets of the first plurality of droplets contain greater than or equal to 5 and less than or equal to 10⁸ distinct nucleic acid sequences. Other ranges are also possible.

Any appropriate fraction of the droplets of the first plurality may include a number of distinct nucleic acids, e.g., described in the preceding paragraph. In some embodiments, greater than or equal to 1%, greater than or equal to 5%, greater than or equal to 10%, greater than or equal to 25%, greater than or equal to 50%, or more of the droplets of a plurality of droplets contains a number of distinct nucleic acids described above. In some embodiments, less than or equal to 100%, less than or equal to 99%, less than or equal to 95%, less than or equal to 75%, less than or equal to 50%, less than or equal to 25%, or less of the droplets of a plurality of droplets contains a number of distinct nucleic acids described above. Combinations of these ranges are possible. For example, in some embodiments, greater than or equal to 1% and less than or equal to 100% of the droplets of a plurality of droplets may contain a number of distinct nucleic acids described above. Other ranges are also possible.

In one set of embodiments, a sample containing nucleic acids may be contained within a plurality of droplets, e.g., contained within a suitable carrying fluid. The nucleic acids may be present during formation of the droplets, and/or added to the droplets after formation. Any suitable method may be chosen to create droplets, and a wide variety of different droplet makers and techniques for forming droplets will be known to those of ordinary skill in the art. For example, a junction of channels may be used to create the droplets. The junction may be, for instance, a T-junction, a Y-junction, a channel-within-a-channel junction (e.g., in a coaxial arrangement, or comprising an inner channel and an outer channel surrounding at least a portion of the inner channel), a cross (or “X”) junction, a flow-focusing junction, or any other suitable junction for creating droplets. See, for example, International Patent Application No. PCT/US2004/010903, filed Apr. 9, 2004, entitled “Formation and Control of Fluidic Species,” by Link, et al., published as WO 2004/091763 on Oct. 28, 2004, or International Patent Application No. PCT/US2003/020542, filed Jun. 30, 2003, entitled “Method and Apparatus for Fluid Dispersion,” by Stone, et al., published as WO 2004/002627 on Jan. 8, 2004, each of which is incorporated herein by reference in its entirety.

In certain embodiments, nucleic acids may be added to droplet after the droplet has been formed, e.g., through picoinjection or other methods such as those discussed in Int. Pat. Apl. Pub. No. WO 2010/151776, entitled “Fluid Injection” (incorporated herein by reference), through fusion of the droplets with droplets containing the nucleic acids, or through other techniques known to those of ordinary skill in the art.

The nucleic acids may be natural nucleic acids, such as DNA or RNA. In some cases, a nucleic acid that activates the target substrate may be present at very low concentrations. For instance, a nucleic acid that activates the target substrate may be present in a droplet containing other nucleic acids at a concentration of 1:10³, 1:10⁴, 1:10⁵, 1:10⁶, 1:10⁷, 1:10⁸, or even lower concentrations, versus the total number of nucleic acids in the droplet. In some embodiments, a nucleic acid that activates the target substrate may be totally non-existent in at least some droplets of the plurality. On the other hand, in some embodiments, a particular nucleic acid that activates the target substrate is present in multiple droplets of the plurality, such that multiple droplets contain activity of the target substrate resulting from action of a single nucleic acid common to the multiple droplets.

It should be understood that the nucleic acid may activate the target substrate directly (e.g., by direct action on the target substrate) or indirectly (e.g., by encoding a protein that, under the proper conditions, can be produced using the nucleic acid in order to activate the target substrate).

The target substrate may also be included in the droplet. As with the nucleic acids described above, the target substrate may be present in the droplets initially, or may be introduced after their formation (e.g., using an aforementioned technique, such as picoinjection).

Some aspects relate to mRNAs produced by “in vitro transcription” or IVT. IVT methods produce (e.g., synthesize) an RNA transcript (e.g., mRNA transcript) by contacting a DNA template (e.g., an input DNA) with an RNA polymerase (e.g., a T7 RNA polymerase, a T7 RNA polymerase variant, etc.) under conditions that result in the production of the RNA transcript. IVT conditions typically employ a DNA template containing a promoter, nucleoside triphosphates, a buffer system that includes dithiothreitol (DTT) and magnesium ions, and an RNA polymerase. The exact conditions used in the transcription reaction depend on the amount of RNA needed for a specific application.

Some aspects relate to proteins produced by “in vitro transcription and translation” or IVTT. IVTT may be performed such that transcription and translation occur simultaneously for a given complex, or at least so that transcription and translation occur simultaneously within the same solution. IVTT methods may produce (e.g., synthesize) a polypeptide (e.g., a protein) by contacting an mRNA produced in vitro with a ribosome under conditions that result in the production of the protein. IVTT conditions typically employ a DNA template containing a promoter, a ribosome binding site, nucleoside triphosphates, a buffer system that includes dithiothreitol (DTT) and magnesium ions, an RNA polymerase, and a ribosome. The exact conditions used in the IVTT reaction may depend on the amount of protein needed for a specific application.

IVT and IVTT may be performed in droplets, depending on the embodiment, such that IVT or IVTT may be performed as part of a method described herein. In some embodiments, the first plurality of droplets comprises a nucleic acid sequence that encodes a mRNA or protein that activates the target substrate. In some embodiments, a method comprises translating proteins from one or more nucleic acids (e.g., mRNA molecules transcribed from nucleic acids of a library) present within the first plurality of droplets. As a corollary, according to some embodiments, proteins are translated prior to determining the one or more droplets of the first plurality of droplets that contain activity of the target substrate. Transcription and translation within a plurality of droplets may have certain advantages. For example, transcription and translation within a plurality of droplets may ensure that activity of a target substrate within a droplet is associated exclusively with the nucleic acids present within that droplet, and does not result from nucleic acids that are not present within that droplet. The library may be used to transcribe any appropriate proteins or peptides, including enzymes or antigenic determinants.

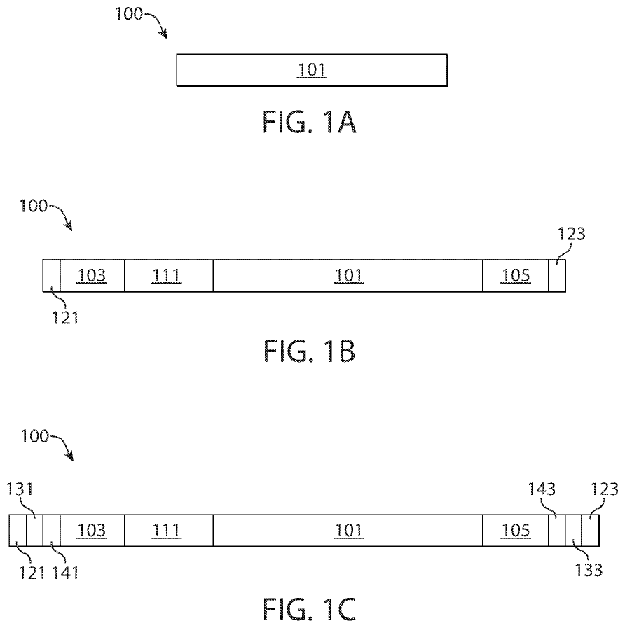

As illustrative non-limiting examples, FIG. 1A presents a schematic illustration of a nucleic acid 100 comprising a library nucleic acid 101. The library nucleic acid 101 may be inserted into a vector for IVTT. For example, FIG. 1B presents a schematic illustration of a nucleic acid 100 comprising library nucleic acid 101, promotor 103, and ribosome binding site 111. Nucleic acid 101 may further comprise a terminator 105.

In some embodiments, a leading and/or a trailing nucleic acid sequence may be used as a priming site for a primer. For example, referring again to FIG. 1B a nucleic acid 100 may comprise priming sites 121 and 123 for primers. To amplify the nucleic acid (e.g., during performance of a method described above), primers may be added to a solution that recognize priming sites 121 and 123. In some embodiments, multiple priming sites may be used for successive iterations of amplification steps. For example, FIG. 1C presents a schematic illustration of nucleic acid 100 comprises a first pair of priming sites 121 and 123 for a first set of primers, used during a first amplification step; a second pair of priming sites 131 and 133 for a second set of primers, used during a second amplification step; and a third pair of priming sites 141 and 143 for a third set of primers, used during a third amplification step.

Nucleic acids contained within activity droplets containing activity of a target substrate may be amplified within the activity droplets. However, amplification may cause multiple nucleic acids to be amplified, which can make it difficult to identify the nucleic acids that activated the target substrate.

Accordingly, in some cases, the amplified nucleic acids may be separated into another plurality of droplets. For instance, in one set of embodiments, the activity droplets may be broken and their contents pooled together, e.g., to create a pool of nucleic acids. The nucleic acids may be amplified (e.g., in the plurality of activity droplets, or in the pool). The pool of amplified nucleic acids may then be made into a new plurality of droplets for further analysis. Alternatively or additionally, the nucleic acids of the first plurality may be made into a second plurality of droplets and amplified within the second plurality of droplets. In some cases, if the pool of amplified nucleic acids has been sufficiently limited by sorting of the activity droplets, the amplified nucleic acids may be sequenced or determined (e.g., qualitatively or quantitatively) as discussed below.

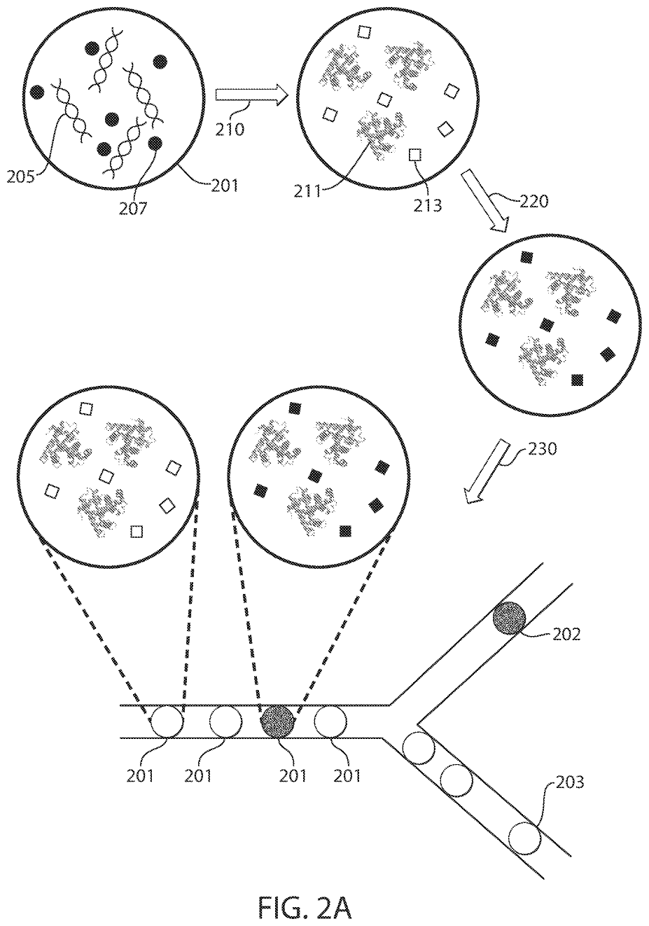

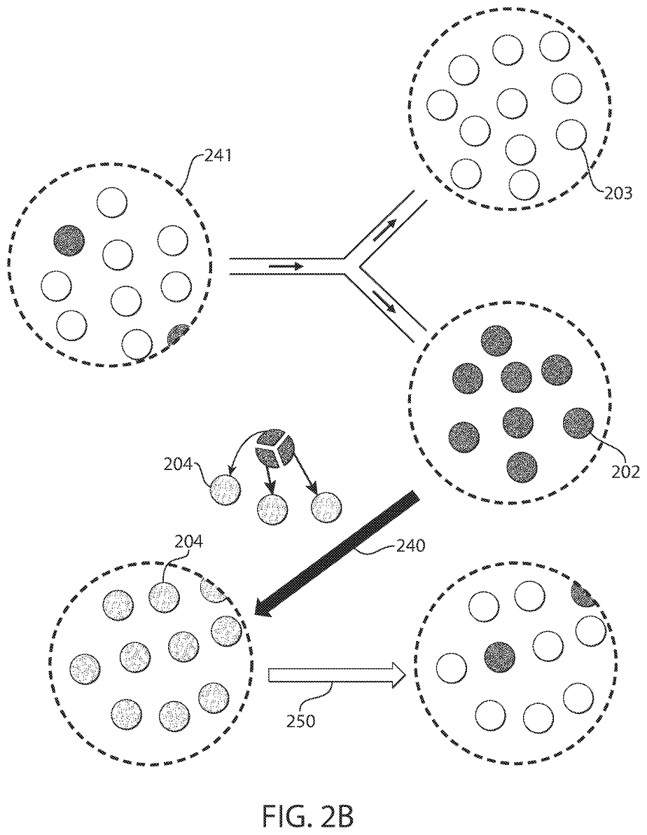

FIG. 2A presents a non-limiting, schematic illustration of an example method described herein. Initially, a droplet 201 comprises nucleic acids 205 and optional other reagents 207. Nucleic acids of the droplet are transcribed and translated in step 210, as described below, to produce proteins 211. Target substrate 213 is included in droplet 201, and is inactive, as indicated in FIG. 2A by the fact that target substrate 213 is a white square. In some droplets 201, target substrate 213 is activated (step 220) by at least some proteins translated from the library, as indicated by the color change of the target substrate to a black square. Droplets 201 may then be sorted (step 230) into activity droplets 202 and non-activity droplets 203. Nucleic acids of the activity droplets can then be amplified and otherwise processed as described above.

FIG. 2B presents a non-limiting, schematic illustration of a portion of a method such as described herein. Initially, a plurality of droplets (some of which are shown in dashed circle 241) are sorted into a plurality of activity droplets 202 and a plurality of non-activity droplets 203, also shown within dashed circles 241 in FIG. 2A. Nucleic acids from activity droplets 202 can then be incorporated into a plurality of new droplets 204 (step 240). The nucleic acids can be amplified in activity droplets 202, or can be amplified after separation into new droplets 204. The new droplets 204 may contain fewer nucleic acids than the activity droplets 202 as a result of this separation. Optionally, step 250 may be performed, wherein new droplets 204 are treated (e.g., subjected to in vitro translation) to produce new activity droplets 202 and new non-activity droplets 203.

In some embodiments, a plurality of droplets containing amplified nucleic acids may be further refined by iterating one or more of the steps described above. For example, a starting plurality of droplets (e.g., comprising amplified nucleic acids of activity droplets belonging to the first plurality of droplets) may be sorted based on activity of a target substrate in the starting plurality of droplets. In some embodiments, activity droplets comprising activity of the target substrate may be separated from the remaining droplets of the starting plurality of droplets. Nucleic acids of the activity droplets may be separated into a new plurality of droplets. The nucleic acids of the activity droplets may be amplified before or after separation into the new plurality of droplets. Referring again to FIG. 2B, in some embodiments, sorting and separating steps as shown are iterated to identify nucleic acids associated with activity droplets of successive pluralities of droplets.

In some embodiments, the new plurality of droplets includes a smaller number of nucleic acids per droplet than the starting plurality of droplets. For example, in some embodiments, an average number of nucleic acids in a droplet of a new plurality of droplets is less than or equal to 10⁻¹, less than or equal to 10⁻², less than or equal to 10⁻³, less than or equal to 10⁻⁴, or less than or equal to 10⁻⁵ or less times an average number of nucleic acids in a droplet of a starting plurality of droplets. In some embodiments, an average number of nucleic acids in a droplet of a new plurality of droplets is greater than or equal to 10⁻⁶, greater than or equal to 10⁻⁵, greater than or equal to 10⁻⁴ or more times an average number of nucleic acids in a droplet of a starting plurality of droplets. Combinations of these ranges are possible. For example, in some embodiments, an average number of nucleic acids in a droplet of a new plurality of droplets is greater than or equal to 10⁻⁶ and less than or equal to 10⁻¹ times an average number of nucleic acids in a droplet of a starting plurality of droplets. Other ranges are also possible.

The steps above may be iterated in any of a variety of suitable orders, and for any of a variety of suitable numbers of iterations. According to some embodiments, a method comprises repeating steps (a)-(c), where: step (a) is determining one or more droplets of a starting plurality of droplets that contain activity of a target substrate; step (b) is separating nucleic acids from the one or more droplets of the starting plurality of droplets into a new plurality of droplets; and step (c) is amplifying the new nucleic acids within the second plurality of droplets. Steps (a)-(c) may be repeated one or more times, depending on the desired attributes of an ultimate new plurality of droplets. For example, in some embodiments, a method comprises repeating steps (a)-(c) until greater than or equal to 10%, 25%, 50%, 75%, 90%, 95%, 99%, or more of the droplets of the new plurality of droplets comprise less than or equal to 1 distinct nucleic acid sequence.

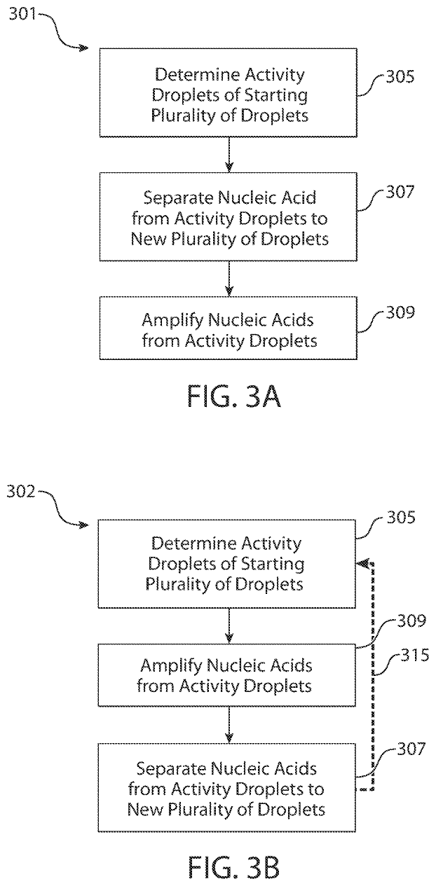

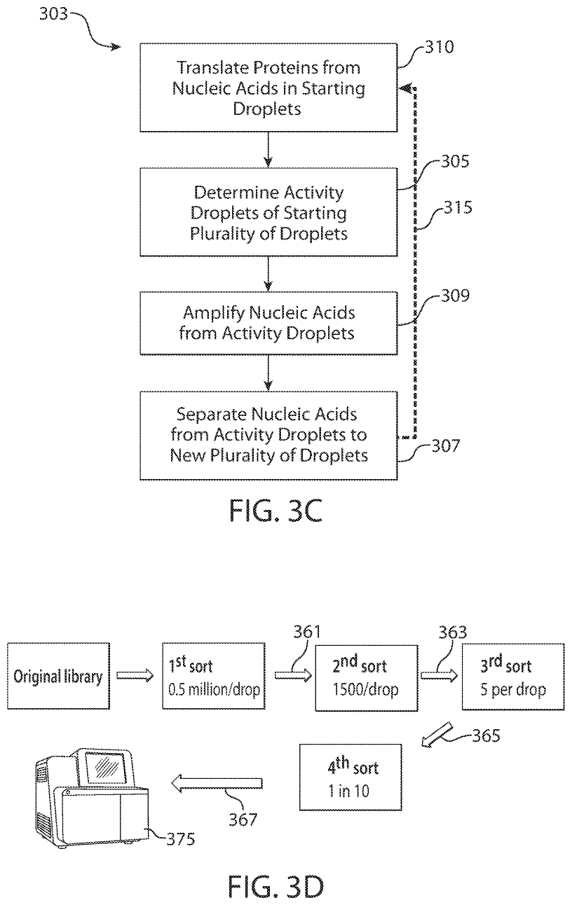

FIG. 3A presents a non-limiting, schematic illustration of a method 301 of refining a nucleic acid library. The method comprises a first step 305 of determining activity droplets of a starting plurality of droplets. The method further comprises a step 307 of separating nucleic acids from the activity droplets to a new plurality of droplets, and a step 309 of amplifying nucleic acids from activity droplets. Although in the method of FIG. 3A step 309 occurs after step 307, this is not necessary. FIG. 3B presents a non-limiting schematic illustration of a method 302 of refining a nucleic acid library. Method 302 comprises steps 305, 307, and 309 as shown in FIG. 3A, but in this example step 309 is performed before step 307. As indicated by dashed arrow 315, methods such as method 302 may optionally be iterated, with the new plurality of droplets acting as a starting plurality of droplets during an iterated step 305 of determining activity droplets within the starting plurality. FIG. 3C presents a non-limiting, schematic illustration of a method 303 of refining a nucleic acid library. Method 303 is similar to method 302 of FIG. 3B. However, method 303 further comprises step 310 of translating proteins from nucleic acids in the starting droplets (e.g., using in vitro translation). The translations step is not necessary to all embodiments, but may be useful for screening of libraries that can express active proteins.

FIG. 3D presents a non-limiting, schematic illustration of a method comprising iterative refinement of a nucleic acid library. In a first step 361, an original library is included in a plurality of droplets at a concentration of 5×10⁵ nucleic acids per droplet. In a second step 363, activity droplets are sorted from the first plurality and are used to form a second plurality of droplets including 1,500 nucleic acids per droplet. In a third step 367, activity droplets are sorted from the second plurality and are used to form a third plurality of droplets including 5 nucleic acids per droplet. In a fourth step 367, activity droplets are sorted from the third plurality and are used to form a fourth plurality of droplets including 1 nucleic acid per 10 droplets. Finally, in a fifth step 369, the droplets are sent to instrument 375 for sequencing.

A fluidic system may be used to perform some or all of the method steps described above. In one aspect of the present invention, emulsions are formed by flowing two, three, or more fluids through a system of channels of a fluidic system. The fluidic system may be or comprise an article. The system or article may be a microfluidic system or article. “Microfluidic,” as used herein, refers to a device, apparatus or system including at least one fluid channel having a cross-sectional dimension (measured perpendicular to the direction of fluid flow) of less than about 1 millimeter (mm), and in some cases, a ratio of length to largest cross-sectional dimension of at least 3:1.

A “channel,” as used herein, means a feature on or in a system or article that at least partially directs flow of a fluid. The channel can have any cross-sectional shape (circular, oval, triangular, irregular, square or rectangular, or the like) and can be covered or uncovered. One or more of the channels may (but not necessarily), in cross section, have a height that is substantially the same as a width at the same point.

In embodiments where it is completely covered, at least one portion of the channel can have a cross-section that is completely enclosed, or the entire channel may be completely enclosed along its entire length with the exception of its inlet(s) and/or outlet(s). A channel may also have an aspect ratio (length to average cross sectional dimension) of at least 2:1, more typically at least 3:1, 5:1, 10:1, 15:1, 20:1, or more. An open channel generally will include characteristics that facilitate control over fluid transport, e.g., structural characteristics (an elongated indentation) and/or physical or chemical characteristics (hydrophobicity vs. hydrophilicity) or other characteristics that can exert a force (e.g., a containing force) on a fluid. The fluid within the channel may partially or completely fill the channel. In some cases where an open channel is used, the fluid may be held within the channel, for example, using surface tension (i.e., a concave or convex meniscus).

The channel may be of any size, for example, having a largest dimension perpendicular to fluid flow of less than about 5 mm or 2 mm, or less than about 1 mm, or less than about 500 microns, less than about 200 microns, less than about 100 microns, less than about 60 microns, less than about 50 microns, less than about 40 microns, less than about 30 microns, less than about 25 microns, less than about 10 microns, less than about 3 microns, less than about 1 micron, less than about 300 nm, less than about 100 nm, less than about 30 nm, or less than about 10 nm. In some cases the dimensions of the channel may be chosen such that fluid is able to freely flow through the article or substrate. The dimensions of the channel may also be chosen, for example, to allow a certain volumetric or linear flowrate of fluid in the channel. Of course, the number of channels and the shape of the channels can be varied by any method known to those of ordinary skill in the art. In some cases, more than one channel or capillary may be used. For example, two or more channels may be used, where they are positioned inside each other, positioned adjacent to each other, positioned to intersect with each other, etc.

The fluidic droplets within the channels may have a cross-sectional dimension smaller than about 100% of an average cross-sectional dimension of the channel, and in certain embodiments, smaller than about 90%, smaller than about 80%, about 70%, about 60%, about 50%, about 40%, about 30%, about 20%, about 10%, about 5%, about 3%, about 1%, about 0.5%, about 0.3%, about 0.1%, about 0.05%, about 0.03%, or about 0.01% of the average cross-sectional dimension of the channel.

During use, at least some processing of the droplets may be performed on an article. Thus, in some embodiments, an article comprises at least some of a plurality of droplets described above. For example, the article may comprise all droplets of a plurality of droplets. The droplets may be fluidically connected to one or more reservoirs of the fluidic system (e.g., to a pool used to form droplets, to a hydrophobic fluid used to form droplets, to a supply of a target substrate, to a supply of a detection agent, to a supply of in vitro transcription and translation reagents, or any of a variety of other fluids described herein) via the article. For example, the droplets may be connected to one or more reservoirs of a fluidic system via the microchannel.

In some embodiments, the fluidic system comprises one or more additional components, such as a pressure source (for example, a pump), a detection tool (e.g., a sensor that may be used to detect fluorescence, luminescence, and/or colorimetric changes resulting from activity of a target substrate); and/or a waste stream.

Although activity of a target substrate may be directly detectable (for example, when the activity causes a change in an optical property of a target substrate, such as fluorescence, luminescence, or a colorimetric change). However, in some embodiments, direct detection of target substrate activity is difficult or impossible. It may be advantageous, particularly when direct activity of a target substrate is difficult or impossible to detect, to include a detection agent for the purpose of detecting activity of the target substrate.

Any of a variety of types of detection agents may be used. In some embodiments, a detection agent is an indirect proxy for activity of a target substrate. For example, the detection agent may be an indicator that is configured to experience a signal change in the presence of target substrate activity. The detection agent may be configured to produce a signal (e.g., an optical signal such as fluorescence, luminescence, or a colorimetric signal) when it encounters an activated target substrate. According to some embodiments, and activated target substrate inhibits a signal produced by a detection agent in the absence of the target substrate. For example, a detection agent may be configured to react with an activated target substrate via a reaction that consumes the detection agent, or that chemically modifies a detection agent to render it undetectable.

In some cases, the nucleic acids within the droplets may be amplified. This may be useful, for example, to produce a larger number or concentration of nucleic acids, e.g., for subsequent analysis, sequencing, or the like. Those of ordinary skill in the art will be familiar with various amplification methods that can be used, including, but not limited to, polymerase chain reaction (PCR), reverse transcriptase (RT), PCR amplification, in vitro transcription amplification (IVT), multiple displacement amplification (MDA), or quantitative real-time PCR (qPCR).

In some cases, the nucleic acids may be amplified within the droplets. Nucleic acid amplification within the droplets may allow amplification to occur “evenly” in some embodiments, e.g., such that the distribution of nucleic acids is not substantially changed after amplification, relative to before amplification. For example, according to certain embodiments, the nucleic acids within a plurality of droplets may be amplified such that the number of nucleic acid molecules for each type of nucleic acid may have a distribution such that, after amplification, no more than about 5%, no more than about 2%, or no more than about 1% of the nucleic acids have a number less than about 90% (or less than about 95%, or less than about 99%) and/or greater than about 110% (or greater than about 105%, or greater than about 101%) of the overall average number of amplified nucleic acid molecules per droplet. In some embodiments, the nucleic acids within the droplets may be amplified such that each of the nucleic acids that are amplified can be detected in the amplified nucleic acids, and in some cases, such that the mass ratio of the nucleic acid to the overall nucleic acid population changes by less than about 50%, less than about 25%, less than about 20%, less than about 15%, less than about 10%, or less than about 5% after amplification, relative to the mass ratio before amplification.

In some cases, certain primers are contained within the droplets to promote amplification. Such primers may be present during formation of the droplets, and/or added to the droplets after formation of the droplets. It should be noted that the manner in which the primers are added to the droplets may be the same or different from the manner in which the nucleic acids are added to the droplets.

In certain embodiments, a plurality of different types of primers may be added to the droplets. Different primers may be distinguishable due to their having different sequences, and/or may be able to amplify different potential targets. In some cases, at least 2, at least 3, at least 5, at least 10, at least 15, at least 20, at least 25, at least 30, at least 40, at least 50, at least 60, at least 75, at least 100, at least 150, at least 200, at least 300, at least 400, at least 500, at least 1,000, at least 2,000, at least 3,000, at least 5,000, or at least 10,000, etc., different primers may be used. This may allow, for example, a variety of different target nucleic acids to be amplified within different droplets.

Examples of techniques for forming droplets include those described above. Examples of techniques for introducing primers after droplet formation include picoinjection or other methods such as those discussed in Int. Pat. Apl. Pub. No. WO 2010/151776, incorporated herein by reference, through fusion of the droplets with droplets containing primers, or the like. Other such techniques for either of these include, but are not limited to, any of those techniques described herein.

The primers may be present within the droplets at any suitable density. For example, the primers may have a density of greater than or equal to 0.1 micromolar, greater than or equal to 0.3 micromolar, greater than or equal to 0.5 micromolar, greater than or equal to 0.8 micromolar, greater than or equal to 1 micromolar, greater than or equal to 5 micromolar, or more. In some embodiments, the primers have a density of less than or equal to 100 micromolar, less than or equal to 50 micromolar, less than or equal to 20 micromolar, less than or equal to 10 micromolar, less than or equal to 5 micromolar, less than or equal to 1 micromolar, or less. Combinations of these ranges are also possible (e.g., greater than or equal to 0.1 micromolar and less than or equal to 100 micromolar). Other ranges are also possible. The density may be independent of the density of target nucleic acids. In some cases, an excess of primers are used, e.g., such that the target nucleic acids controls the reaction. For instance, if a large excess of primers are used, then substantially of the droplets will contain primer (regardless of whether or not the droplets also contain target nucleic acids). For example, in certain embodiments, at least about 50%, at least about 60%, at least about 70%, at least about 80%, at least about 90%, at least about 95%, at least about 97%, at least about 98%, or at least about 99% of the droplets may contain at least one amplification primer.

Droplets containing both primer and a target nucleic acid may be treated to cause amplification of the nucleic acid to occur. This may allow a large amount or concentration of the target nucleic acids to be produced, e.g., without substantially altering the distribution of nucleic acids. In some cases, the primers are selected to allow substantially all, or only some, of the target nucleic acids suspected of being present to be amplified.

As examples, PCR (polymerase chain reaction) or other amplification techniques may be used to amplify nucleic acids, e.g., contained within droplets. Typically, in PCR reactions, the nucleic acids are heated (e.g., to a temperature of at least about 50° C., at least about 70° C., or least about 90° C. in some cases) to cause dissociation of the nucleic acids into single strands, and a heat-stable DNA polymerase (such as Taq polymerase) is used to amplify the nucleic acid. This process is often repeated multiple times to amplify the nucleic acids.

Thus, in one set of embodiments, PCR amplification may be performed within the droplets. For example, the droplets may contain a polymerase (such as Taq polymerase), and DNA nucleotides (deoxyribonucleotides), and the droplets may be processed (e.g., via repeated heated and cooling) to amplify the nucleic acid within the droplets. Suitable reagents for PCR or other amplification techniques, such as polymerases and/or deoxyribonucleotides, may be added to the droplets during their formation, and/or afterwards (e.g., via merger with droplets containing such reagents, and/or via direct injection of such reagents, e.g., contained within a fluid). Various techniques for droplet injection or merger of droplets will be known to those of ordinary skill in the art. See, e.g., U.S. Pat. Apl. Pub. No. 2012/0132288, incorporated herein by reference. In some embodiments, primers may be added to the droplets, or the primers may be present on one or more of the nucleic acids within the droplets. Those of ordinary skill in the art will be aware of suitable primers, many of which can be readily obtained commercially.

In one set of embodiments, at least some of the primers may be distinguished, for example, using distinguishable fluorescent tags, barcodes, or other suitable identification tags. Examples of barcodes that can be contained within droplets include, but are not limited to, those described in U.S. Pat. Apl. Pub. No. 2018-0304222 or Int. Pat. Apl. Pub. No. WO 2015/164212, each incorporated herein by reference.

The nucleic acids may be amplified to any suitable extent. The degree of amplification may be controlled, for example, by controlling factors such as the temperature, cycle time, or amount of enzyme and/or deoxyribonucleotides contained within the droplets. For instance, in some embodiments, a population of droplets may have at least about 50,000, at least about 100,000, at least about 150,000, at least about 200,000, at least about 250,000, at least about 300,000, at least about 400,000, at least about 500,000, at least about 750,000, at least about 1,000,000 or more molecules of the amplified nucleic acid per droplet.

In one set of embodiments, the droplets are broken down after amplification, e.g., to allow the amplified nucleic acids to be pooled together. A wide variety of methods for “breaking” or “bursting” droplets are available to those of ordinary skill in the art. For example, droplets contained in a carrying fluid may be disrupted using techniques such as mechanical disruption, chemical disruption, or ultrasound. Droplets may also be disrupted using chemical agents or surfactants, for example, 1H,1H,2H,2H-perfluorooctanol.

In some embodiments, a method comprises purifying nucleic acids (e.g., nucleic acids pooled from a plurality of droplets). Purification may be used, for example, to extract the nucleic acids from unwanted reagents used in earlier steps. For example, purification may be used to extract the nucleic acids from proteins transcribed therefrom. Any of a variety of appropriate techniques may be used to purify the nucleic acids. For example, the nucleic acids may be purified using any of a variety of suitable methods, such as column- or gel-based methods (including electrophoretic and centrifuge-based methods). For example, nucleic acids may be purified using a PCR clean-up kit.

After refinement of activity-producing nucleic acids, the nucleic acids may optionally be determined and/or sequenced, e.g., using techniques such as those described herein. In some embodiments, the droplets may be burst and the nucleic acids may be combined to facilitate determination and/or sequencing, although in some cases, the determination and/or sequencing may occur within the droplets.

In addition, in certain embodiments, the pool of amplified nucleic acids may be sequenced using droplet-based techniques, e.g., droplet-based PCR. For example, in some cases, the amplified nucleic acids may be collected into droplets and the droplets exposed to certain primers. In some cases, the amplified nucleic acids may be collected into droplets at relatively low concentrations, e.g., such that the droplets may, on the average, contain less than 1 nucleic acid per droplet, as described herein. In addition, in certain embodiments, the droplets may be divided into different groups of droplets, which are exposed to different primers. For instance, the droplets may be divided into at least 5, 10, 30, 100, etc. groups, which are exposed to various primers, e.g., in different spatial locations, to determine whether a target nucleic acid was present in the sample. However, it should be understood that in other embodiments, the amplified nucleic acids may be present at relatively higher concentrations, e.g., at least 1 nucleic acid per droplet or at least 1 target per droplet. In some cases, more than one primer or one amplicon may be present within a droplet.

Examples of methods for determining and/or sequencing nucleic acids include, but are not limited to, chain-termination sequencing, sequencing-by-hybridization, Maxam-Gilbert sequencing, dye-terminator sequencing, chain-termination methods, Massively Parallel Signature Sequencing (Lynx Therapeutics), polony sequencing, pyrosequencing, sequencing by ligation, ion semiconductor sequencing, DNA nanoball sequencing, single-molecule real-time sequencing (e.g., Pacbio sequencing), nanopore sequencing, Sanger sequencing, digital RNA sequencing (“digital RNA-seq”), Illumina sequencing, etc. In some cases, a microarray, such as a DNA microarray, may be used, for example, to determine, or to sequence, a nucleic acid. In some cases, the pool of amplified nucleic acids may be determined or identified, e.g., without any sequencing.

Additional details regarding systems and methods for manipulating droplets in a microfluidic system follow, in accordance with certain aspects. For example, various systems and methods for screening and/or sorting droplets are described in U.S. patent application Ser. No. 11/360,845, filed Feb. 23, 2006, entitled “Electronic Control of Fluidic Species,” by Link, et al., published as U.S. Patent Application Publication No. 2007/000342 on Jan. 4, 2007, incorporated herein by reference. As a non-limiting example, in some aspects, by applying (or removing) a first electric field (or a portion thereof), a droplet may be directed to a first region or channel; by applying (or removing) a second electric field to the device (or a portion thereof), the droplet may be directed to a second region or channel; by applying a third electric field to the device (or a portion thereof), the droplet may be directed to a third region or channel; etc., where the electric fields may differ in some way, for example, in intensity, direction, frequency, duration, etc.

As mentioned, certain embodiments comprise a droplet contained within a carrying fluid. For example, there may be a first phase forming droplets contained within a second phase, where the surface between the phases comprises one or more proteins. For example, the second phase may comprise oil or a hydrophobic fluid, while the first phase may comprise water or another hydrophilic fluid (or vice versa). It should be understood that a hydrophilic fluid is a fluid that is substantially miscible in water and does not show phase separation with water at equilibrium under ambient conditions (typically 25° C. and 1 atm). Examples of hydrophilic fluids include, but are not limited to, water and other aqueous solutions comprising water, such as cell or biological media, ethanol, salt solutions, saline, blood, etc. In some cases, the fluid is biocompatible.

Similarly, a hydrophobic fluid is one that is substantially immiscible in water and will show phase separation with water at equilibrium under ambient conditions. As previously discussed, the hydrophobic fluid is sometimes referred to by those of ordinary skill in the art as the “oil phase” or simply as an oil. Non-limiting examples of hydrophobic fluids include oils such as hydrocarbons oils, silicon oils, fluorocarbon oils, organic solvents, perfluorinated oils, perfluorocarbons such as perfluoropolyether, etc. Additional examples of potentially suitable hydrocarbons include, but are not limited to, light mineral oil (Sigma), kerosene (Fluka), hexadecane (Sigma), decane (Sigma), undecane (Sigma), dodecane (Sigma), octane (Sigma), cyclohexane (Sigma), hexane (Sigma), or the like. Non-limiting examples of potentially suitable silicone oils include 2 cst polydimethylsiloxane oil (Sigma). Non-limiting examples of fluorocarbon oils include FC3283 (3M), FC40 (3M), Krytox GPL (Dupont), etc. In addition, other hydrophobic entities may be contained within the hydrophobic fluid in some embodiments. Non-limiting examples of other hydrophobic entities include drugs, immunologic adjuvants, or the like.

Thus, the hydrophobic fluid may be present as a separate phase from the hydrophilic fluid. In some embodiments, the hydrophobic fluid may be present as a separate layer, although in other embodiments, the hydrophobic fluid may be present as individual fluidic droplets contained within a continuous hydrophilic fluid, e.g. suspended or dispersed within the hydrophilic fluid. This is often referred to as an oil/water emulsion. The droplets may be relatively monodisperse, or be present in a variety of different sizes, volumes, or average diameters. In some cases, the droplets may have an overall average diameter of less than about 1 mm, or other dimensions as discussed herein. In some cases, a surfactant may be used to stabilize the hydrophobic droplets within the hydrophilic liquid, for example, to prevent spontaneous coalescence of the droplets. Non-limiting examples of surfactants include those discussed in U.S. Pat. Apl. Pub. No. 2010/0105112, incorporated herein by reference. Other non-limiting examples of surfactants include Span80 (Sigma), Span80/Tween-20 (Sigma), Span80/Triton X-100 (Sigma), Abil EM90 (Degussa), Abil we09 (Degussa), polyglycerol polyricinoleate “PGPR90” (Danisco), Tween-85, 749 Fluid (Dow Corning), the ammonium carboxylate salt of Krytox 157 FSL (Dupont), the ammonium carboxylate salt of Krytox 157 FSM (Dupont), or the ammonium carboxylate salt of Krytox 157 FSH (Dupont). In addition, the surfactant may be, for example, a peptide surfactant, bovine serum albumin (BSA), or human serum albumin.

The droplets may have any suitable shape and/or size. In some cases, the droplets may be microfluidic, and/or have an average diameter of less than about 1 mm. For instance, the droplet may have an average diameter of less than about 1 mm, less than about 700 micrometers, less than about 500 micrometers, less than about 300 micrometers, less than about 100 micrometers, less than about 70 micrometers, less than about 50 micrometers, less than about 30 micrometers, less than about 10 micrometers, less than about 5 micrometers, less than about 3 micrometers, less than about 1 micrometer, etc. The average diameter may also be greater than about 1 micrometer, greater than about 3 micrometers, greater than about 5 micrometers, greater than about 7 micrometers, greater than about 10 micrometers, greater than about 30 micrometers, greater than about 50 micrometers, greater than about 70 micrometers, greater than about 100 micrometers, greater than about 300 micrometers, greater than about 500 micrometers, greater than about 700 micrometers, or greater than about 1 mm in some cases. Combinations of any of these are also possible; for example, the diameter of the droplet may be between about 1 mm and about 100 micrometers. The diameter of a droplet, in a non-spherical droplet, may be taken as the diameter of a perfect mathematical sphere having the same volume as the non-spherical droplet.

In some embodiments, the droplets may be of substantially the same shape and/or size (i.e., “monodisperse”), or of different shapes and/or sizes, depending on the particular application. In some cases, the droplets may have a homogenous distribution of cross-sectional diameters, i.e., in some embodiments, the droplets may have a distribution of average diameters such that no more than about 20%, no more than about 10%, or no more than about 5% of the droplets may have an average diameter greater than about 120% or less than about 80%, greater than about 115% or less than about 85%, greater than about 110% or less than about 90%, greater than about 10⁵% or less than about 95%, greater than about 103% or less than about 97%, or greater than about 101% or less than about 99% of the average diameter of the microfluidic droplets. Some techniques for producing homogenous distributions of cross-sectional diameters of droplets are disclosed in International Patent Application No. PCT/US2004/010903, filed Apr. 9, 2004, entitled “Formation and Control of Fluidic Species,” by Link, et al., published as WO 2004/091763 on Oct. 28, 2004, incorporated herein by reference. In addition, in some instances, the coefficient of variation of the average diameter of the droplets may be less than or equal to about 20%, less than or equal to about 15%, less than or equal to about 10%, less than or equal to about 5%, less than or equal to about 3%, or less than or equal to about 1%. However, in other embodiments, the droplets may not necessarily be substantially monodisperse, and may instead exhibit a range of different diameters.

Those of ordinary skill in the art will be able to determine the average diameter of a population of droplets, for example, using laser light scattering or other known techniques. The droplets so formed can be spherical, or non-spherical in certain cases. The diameter of a droplet, in a non-spherical droplet, may be taken as the diameter of a perfect mathematical sphere having the same volume as the non-spherical droplet.

In some embodiments, one or more droplets may be created within a channel by creating an electric charge on a fluid surrounded by a liquid, which may cause the fluid to separate into individual droplets within the liquid. In some embodiments, an electric field may be applied to the fluid to cause droplet formation to occur. The fluid can be present as a series of individual charged and/or electrically inducible droplets within the liquid. Electric charge may be created in the fluid within the liquid using any suitable technique, for example, by placing the fluid within an electric field (which may be AC, DC, etc.), and/or causing a reaction to occur that causes the fluid to have an electric charge.

The electric field, in some embodiments, is generated from an electric field generator, i.e., a device or system able to create an electric field that can be applied to the fluid. The electric field generator may produce an AC field (i.e., one that varies periodically with respect to time, for example, sinusoidally, sawtooth, square, etc.), a DC field (i.e., one that is constant with respect to time), a pulsed field, etc. Techniques for producing a suitable electric field (which may be AC, DC, etc.) are known to those of ordinary skill in the art. For example, in one embodiment, an electric field is produced by applying voltage across a pair of electrodes, which may be positioned proximate a channel such that at least a portion of the electric field interacts with the channel. The electrodes can be fashioned from any suitable electrode material or materials known to those of ordinary skill in the art, including, but not limited to, silver, gold, copper, carbon, platinum, copper, tungsten, tin, cadmium, nickel, indium tin oxide (“ITO”), etc., as well as combinations thereof.

In another set of embodiments, droplets of fluid can be created from a fluid surrounded by a liquid within a channel by altering the channel dimensions in a manner that is able to induce the fluid to form individual droplets. The channel may, for example, be a channel that expands relative to the direction of flow, e.g., such that the fluid does not adhere to the channel walls and forms individual droplets instead, or a channel that narrows relative to the direction of flow, e.g., such that the fluid is forced to coalesce into individual droplets. In some cases, the channel dimensions may be altered with respect to time (for example, mechanically or electromechanically, pneumatically, etc.) in such a manner as to cause the formation of individual droplets to occur. For example, the channel may be mechanically contracted (“squeezed”) to cause droplet formation, or a fluid stream may be mechanically disrupted to cause droplet formation, for example, through the use of moving baffles, rotating blades, or the like.

Some embodiments generally relate to systems and methods for fusing or coalescing two or more droplets into one droplet, e.g., where the two or more droplets ordinarily are unable to fuse or coalesce, for example, due to composition, surface tension, droplet size, the presence or absence of surfactants, etc. In certain cases, the surface tension of the droplets, relative to the size of the droplets, may also prevent fusion or coalescence of the droplets from occurring.

As a non-limiting example, two droplets can be given opposite electric charges (i.e., positive and negative charges, not necessarily of the same magnitude), which can increase the electrical interaction of the two droplets such that fusion or coalescence of the droplets can occur due to their opposite electric charges. For instance, an electric field may be applied to the droplets, the droplets may be passed through a capacitor, a chemical reaction may cause the droplets to become charged, etc. The droplets, in some cases, may not be able to fuse even if a surfactant is applied to lower the surface tension of the droplets. However, if the droplets are electrically charged with opposite charges (which can be, but are not necessarily of, the same magnitude), the droplets may be able to fuse or coalesce. As another example, the droplets may not necessarily be given opposite electric charges (and, in some cases, may not be given any electric charge), and are fused through the use of dipoles induced in the droplets that causes the droplets to coalesce. Also, the two or more droplets allowed to coalesce are not necessarily required to meet “head-on.” Any angle of contact, so long as at least some fusion of the droplets initially occurs, is sufficient. See also, e.g., U.S. patent application Ser. No. 11/698,298, filed Jan. 24, 2007, entitled “Fluidic Droplet Coalescence,” by Ahn, et al., published as U.S. Patent Application Publication No. 2007/0195127 on Aug. 23, 2007, incorporated herein by reference in its entirety.

In one set of embodiments, a fluid may be injected into a droplet. The fluid may be microinjected into the droplet in some cases, e.g., using a microneedle or other such device. In other cases, the fluid may be injected directly into a droplet using a fluidic channel as the droplet comes into contact with the fluidic channel. Other techniques of fluid injection are disclosed in, e.g., International Patent Application No. PCT/US2010/040006, filed Jun. 25, 2010, entitled “Fluid Injection,” by Weitz, et al., published as WO 2010/151776 on Dec. 29, 2010; or International Patent Application No. PCT/US2009/006649, filed Dec. 18, 2009, entitled “Particle-Assisted Nucleic Acid Sequencing,” by Weitz, et al., published as WO 2010/080134 on Jul. 15, 2010, each incorporated herein by reference in its entirety.

The following documents are each incorporated herein by reference in its entirety for all purposes: Int. Pat. Apl. Pub. No. WO 2016/168584, entitled “Barcoding System for Gene Sequencing and Other Applications,” by Weitz et al.; Int. Pat. Apl. Pub. No. WO 2015/161223, entitled “Methods and Systems for Droplet Tagging and Amplification,” by Weitz, et al.; U.S. Pat. Apl. Ser. No. 61/980,541, entitled “Methods and Systems for Droplet Tagging and Amplification,” by Weitz, et al.; U.S. Pat. Apl. Ser. No. 61/981,123, entitled “Systems and Methods for Droplet Tagging,” by Bernstein, et al.; Int. Pat. Apl. Pub. No. WO 2004/091763, entitled “Formation and Control of Fluidic Species,” by Link et al.; Int. Pat. Apl. Pub. No. WO 2004/002627, entitled “Method and Apparatus for Fluid Dispersion,” by Stone et al.; Int. Pat. Apl. Pub. No. WO 2006/096571, entitled “Method and Apparatus for Forming Multiple Emulsions,” by Weitz et al.; Int. Pat. Apl. Pub. No. WO 2005/021151, entitled “Electronic Control of Fluidic Species,” by Link et al.; Int. Pat. Apl. Pub. No. WO 2011/056546, entitled “Droplet Creation Techniques,” by Weitz, et al.; Int. Pat. Apl. Pub. No. WO 2010/033200, entitled “Creation of Libraries of Droplets and Related Species,” by Weitz, et al.; U.S. Pat. Apl. Pub. No. 2012-0132288, entitled “Fluid Injection,” by Weitz, et al.; Int. Pat. Apl. Pub. No. WO 2008/109176, entitled “Assay And Other Reactions Involving Droplets,” by Agresti, et al.; and Int. Pat. Apl. Pub. No. WO 2010/151776, entitled “Fluid Injection,” by Weitz, et al.; and U.S. Pat. Apl. Ser. No. 62/072,944, entitled “Systems and Methods for Barcoding Nucleic Acids,” by Weitz, et al.

In addition, the following are incorporated herein by reference in their entireties: U.S. Pat. Apl. Ser. No. 61/981,123 filed Apr. 17, 2014; PCT Pat. Apl. Ser. No. PCT/US2015/026338, filed Apr. 17, 2015, entitled “Systems and Methods for Droplet Tagging”; U.S. Pat. Apl. Ser. No. 61/981,108 filed Apr. 17, 2014; U.S. Pat. Apl. Ser. No. 62/072,944, filed Oct. 30, 2014; PCT Pat. Apl. Ser. No. PCT/US2015/026443, filed on Apr. 17, 2015, entitled “Systems and Methods for Barcoding Nucleic Acids”; U.S. Pat. Apl. Ser. No. 62/106,981, entitled “Systems, Methods, and Kits for Amplifying or Cloning Within Droplets,” by Weitz, et al.; U.S. Pat. Apl. Pub. No. 2010-0136544, entitled “Assay and Other Reactions Involving Droplets,” by Agresti, et al.; U.S. Pat. Apl. Ser. No. 61/981,108, entitled “Methods and Systems for Droplet Tagging and Amplification,” by Weitz, et al.; Int. Pat. Apl. Pub. No. PCT/US2014/037962, filed May 14, 2014, entitled “Rapid Production of Droplets,” by Weitz, et al.; and U.S. Provisional Patent Application Ser. No. 62/133,140, filed Mar. 13, 2015, entitled “Determination of Cells Using Amplification,” by Weitz, et al.

A library of nucleic acids as described herein may prepared by any of a variety of appropriate methods. For example, in some embodiments, a library is prepared by error-prone PCR. However, error-prone PCR is nonrandom; certain codon mutations are more likely than others, and some codon mutations are totally forbidden. By using the systems and methods described below, libraries may be generated by a more uniformly random process, without any forbidden mutations. The libraries may then be screened using one or more of the systems or methods described above. The systems and methods described herein may thereby surpass the performance of conventional methods of library refinement, such as refinement of libraries prepared by error-prone PCR, by screening more functionally robust and diverse nucleic acid libraries, with higher randomness, using fewer steps.

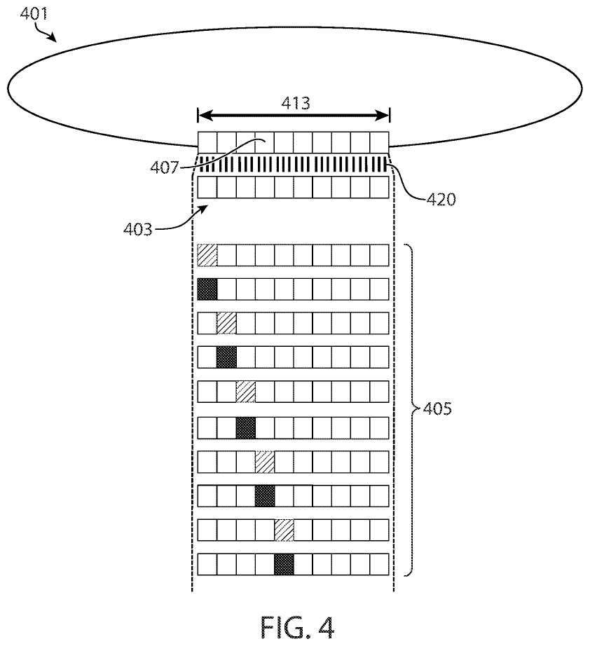

In one aspect, systems and methods of synthesizing a library of nucleic acid sequences are provided. In some cases, a common template nucleic acid and a first plurality of primers may be used to synthesize a first plurality of nucleic acid sequences. In some embodiments, it may be advantageous to use a circular nucleic acid as a common template nucleic acid. However, linear nucleic acids may also be used in other embodiments. FIG. 4 provides a schematic illustration of a non-limiting common template nucleic acid, with nucleic acid 401 represented as a circular nucleic acid. The white rectangles of nucleic acid 401 represent individual codons of nucleic acid 401 that are illustrated for emphasis—but the black curve is also part of nucleic acid 401 that is not shown in detail.

The common template nucleic acid may be selected by a user, and is not limiting. Any suitable template nucleic acid can be used. For example, a template nucleic acid may be chosen because it expresses a protein or other target of interest, of which a favorable improvement is desired. For instance, the template nucleic acid may express an enzyme capable of performing a useful biological or chemical function. In some embodiments, the common template nucleic acid is a circular nucleic acid. In certain embodiments, circular nucleic acids are used, which may be advantageous in some cases because only forward primers need be used. However, the techniques provided herein are not so limited, and noncircular nucleic acids may be used in other embodiments.

In some cases, certain primers are contained within a solution to promote amplification of a nucleic acid. The method may comprise using a plurality of primers to synthesize a plurality of nucleic acids using the common template nucleic acid. For example, FIG. 4 shows plurality 405 of primers that are configured to synthesize a plurality of nucleic acids using nucleic acid 401. In certain embodiments, a plurality of different types of primers may be added to the solution. At least some primers of the plurality of primers may comprise a plurality of consecutive nucleotides that are complementary to a plurality of consecutive nucleotides of the common template nucleic acid. In this way, a primer may be configured to bind to the common template nucleic acid, such that the primer can be used to synthesize a nucleic acid by an amplification technique (e.g., PCR), as described in greater detail below. At least some primers comprising a plurality of consecutive nucleotides of the common template nucleic acid may include at least one codon that is not complementary to a codon of the template nucleic acid. In some embodiments, at least some primers comprising a plurality of consecutive nucleotides complementary to nucleotides of the common template nucleic acid include less than or equal to 2 codons that are not complementary to codons of the common template nucleic acid. For example, the primer may include exactly one codon that is not complementary to a codon of the template nucleic acid. In some embodiments, at least some primers comprising a plurality of consecutive nucleotides complementary to nucleotides of the common template nucleic acid include less than or equal to 4 codons (e.g., less than or equal to 3 codons, less than or equal to 2 codons) that are not complementary to codons of the common template nucleic acid. For example, the primer may include exactly one codon that is not complementary to a codon of the template nucleic acid.

Referring again to FIG. 4, plurality 405 of primers includes ten primers, each comprising 10 codons, represented as individual rectangles. The nine white codons of each primer are complementary to portion 413 of nucleic acid 401; the colored codons represent a codon that is not complementary to codon 407 of nucleic acid 401.

A primer may be configured to bind to a common template nucleic acid, according to some embodiments. For example, referring again to FIG. 4, each white codon of a primer of plurality of primers 405 includes 3 nucleotides that are complementary to 3 nucleotides of portion 413 of nucleic acid 401. In some embodiments, greater than or equal to 5, greater than or equal to 10, greater than or equal to 15, greater than or equal to 20, greater than or equal to 25, greater than or equal to 30, or more consecutive nucleotides of the common template nucleic acid are complimentary to complementary nucleotides of the common template primer. In some embodiments, less than or equal to 100, less than or equal to 75, less than or equal to 50, less than or equal to 40, less than or equal to 30, less than or equal to 25, or fewer consecutive nucleotides of the common template nucleic acid are complimentary to complementary nucleotides of the common template primer. Combinations of these ranges are possible. For example, in some embodiments, greater than or equal to 5 and less than or equal to 100 consecutive nucleotides of the common template nucleic acid are complimentary to complementary nucleotides of the common template primer. Other ranges are also possible.

The primers may be present within the solution at any suitable density. The density may be independent of the density of nucleic acids. In some cases, an excess of primers is used, e.g., such that nucleic acids to be amplified by the primer control the reaction.

Any of a variety of suitable numbers of primers may be used. For instance, referring again to FIG. 4, although 10 primers are shown in plurality 405 of primers, it should, of course, be understood that the plurality may include any appropriate number of primers. In some cases, at least 2, at least 3, at least 5, at least 10, at least 15, at least 20, at least 25, at least 30, at least 40, at least 50, at least 60, at least 75, at least 100, at least 150, at least 200, at least 500, at least 1,000, at least 2,000, at least 5,000, etc., different primers may be used. This may allow, for example, synthesis of a plurality of nucleic acids having one or more codons that differ from a common template nucleic acid. A variety of different nucleic acids may be amplified within different droplets.

The amplification may also be relatively selective, e.g., if library generation is centered on mutations of single, common template nucleic acid, by providing only certain primers. For instance, one or only a relatively small number of primers (e.g., less than or equal to 500, 400, 300, 200, 180, 160, 140, 120, 100, 80, 60, 40, 20, 19, 18, 17, 16, 15, 14, 13, 12, 11, 10, 9, 8, 7, 6, 5, 4, or 3 primers) may be provided in certain embodiments, thereby allowing only specific nucleic acid sequences to be amplified, e.g., within the droplets. In some cases, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 40, at least 60, at least 80, at least 100, at least 120, at least 140, at least 160, at least 180, or at least 200 primers may be present. Combinations of the aforementioned ranges (e.g., at least 2 and less than or equal to 500) are also possible. In some embodiments, exactly one primer is used e.g., for the purpose of amplifying, rather than mutating, circular nucleic acids present in the solution.

As a non-limiting example, primers that allow only certain mutations in a nucleic acid to be amplified may be used during amplification. For instance, a plurality of primers may be used that have relatively small differences, e.g. such that the primers have at least 70%, at least 75%, at least 80%, at least 85%, at least 90%, or at least 95% homology, and/or such that the amplification primers are all substantially complementary to a common template primer, except for no more than 5, 4, 3, 2, or 1 nucleotide differences. For example, referring again to FIG. 4, each primer of plurality 405 of primers includes exactly one non-complementary codon (including no more than 3, 2, or 1 nucleotide differences).

In some embodiments, amplification using multiple pluralities of primers can be used to produce a plurality of nucleic acids. For instance, in certain embodiments, the plurality of primers may represent variations on a common template primer, that is perfectly complementary to a portion of the nucleic acid. For example, in addition to plurality of primers 405, FIG. 4 shows common template primer 403 that is perfectly complementary to portion 413 of nucleic acid 401, as illustrated by nucleotide bonds 420 between portion 413 and common template primer 403. In some embodiments, greater than or equal to 50%, greater than or equal to 60%, greater than or equal to 70%, greater than or equal to 80%, greater than or equal to 90%, greater than or equal to 95%, or more primers of a plurality differ from a common template primer at exactly one codon. In some embodiments, less than or equal to 100%, less than or equal to 95%, or fewer primers of a plurality differ from a common template primer at exactly one codon. Combinations of these ranges are possible. For example, in some embodiments, greater than or equal to 50% and less than or equal to 100% of primers of a plurality differ from a common template primer at exactly one codon. Other ranges are also possible.

In some embodiments, greater than or equal to 50%, greater than or equal to 60%, greater than or equal to 70%, greater than or equal to 80%, greater than or equal to 90%, greater than or equal to 95%, or more primers of a plurality differ from a common template primer at greater than or equal to 1 and less than or equal to 2 codons (e.g., at exactly 2 codons). In some embodiments, less than or equal to 100%, less than or equal to 95%, or fewer primers of a plurality differ from a common template primer at greater than or equal to 1 and less than or equal to 2 codons (e.g., at exactly 2 codons). Combinations of these ranges are possible. For example, in some embodiments, greater than or equal to 50% and less than or equal to 100% of primers of a plurality differ from a common template primer at greater than or equal to 1 and less than or equal to 2 codons (e.g., at exactly 2 codons). Other ranges are also possible. In addition, in some cases, greater than or equal to 50%, greater than or equal to 60%, greater than or equal to 70%, greater than or equal to 80%, greater than or equal to 90%, greater than or equal to 95%, or more primers of a plurality differ from a common template primer at greater than or equal to 1 and less than or equal to 4 codons or less than or equal to 3 codons.

In some embodiments, greater than or equal to 50%, greater than or equal to 60%, greater than or equal to 70%, greater than or equal to 80%, greater than or equal to 90%, greater than or equal to 95%, or more primers of a plurality differ from all other primers of the plurality at no more than two codons. In some embodiments, less than or equal to 100%, less than or equal to 95%, or fewer primers of a plurality differ from all other primers of the plurality at no more than two codons. Combinations of these ranges are possible. For example, in some embodiments, greater than or equal to 50% and less than or equal to 100% of primers of a plurality differ from all other primers of the plurality at no more than two codons. Other ranges are also possible.

Amplification using a plurality of primers including exactly one codon that is not complementary to the common template nucleic acid may thus produce a plurality of nucleic acids that differ from a common template nucleic acid. If the non-complementary codons of the primers occur in the same sequential position, amplification using the primers can produce a plurality of nucleic acids, each of which differs from the template nucleic acid at the non-complementary codon of the primer. Such a reaction may permit development of a library of nucleic acids via point-wise mutations, which may be particularly advantageous for library development, in some embodiments. As a non-limiting example, the common template nucleic acid may be amplified using 19 primers, each of which is non-complementary to the same codon, in order to produce a library of at least 19 nucleic acids (optionally including the common template nucleic acid, and additionally including 19 additional nucleic acids with a one-codon mutation) each of which would be identical, except for the changed codon.

More generally, a plurality of nucleic acids may comprise 19 primers for each codon of a common template primer (based on the 20 naturally-occurring amino acids) in certain embodiments as discussed herein. For example, if a common template primer included 10 codons, the plurality could include up to 190 primers, reflecting every possible single-codon variation of the common template primer. However, in some cases, more or fewer than 19 primers for each codon may be used, for example, to include non-naturally-occurring amino acids or to omit certain amino acids, etc.

An advantageous feature of this approach, in some embodiments, is that a primer differing from the common template nucleic acid at exactly one codon can be amplified either using the common template nucleic acid or using a previously-synthesized nucleic acid. Even if (as is highly probable) a first primer of the plurality was amplified from a nucleic acid produced using second primer of the plurality, the mutation of the second primer would not be incorporated into the synthesized nucleic acid, since both primers have the same length and correspond to the same template primer.

Thus, the number of distinct nucleic acids included in the library may be controlled by the primers used, and generally cannot produce unexpected mutations. Another advantage of this approach is that the library may be used in some embodiments to encode any or every natural amino acid at a given site, and there are no forbidden mutations. Yet another advantage is that mutations known to be detrimental may be deliberately excluded in certain embodiments, since the mutation process may be controlled, at least in part, by the primers used to amplify the nucleic acids.

Multiple mutations can be made simultaneously in some embodiments. For example, two or more pluralities of primers could be used simultaneously in some cases. Primers within a first plurality may be configured to bind to a first portion of the common template nucleic acid (e.g., by corresponding to a first common template primer) and primers within a second plurality may be configured to bind to a second portion of the common template nucleic acid (e.g., by corresponding to a second common template primer, distinct from the first).

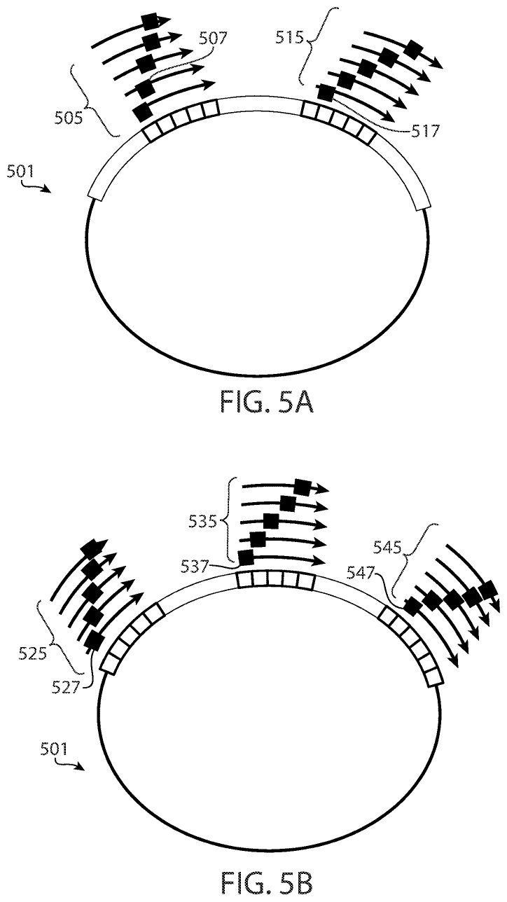

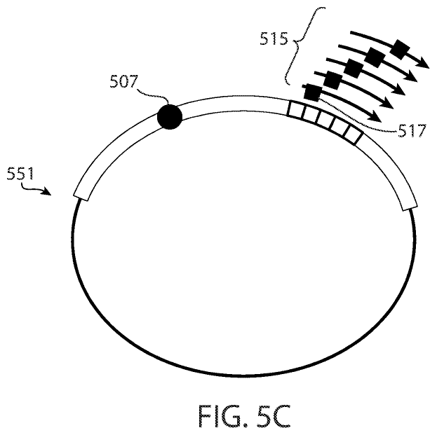

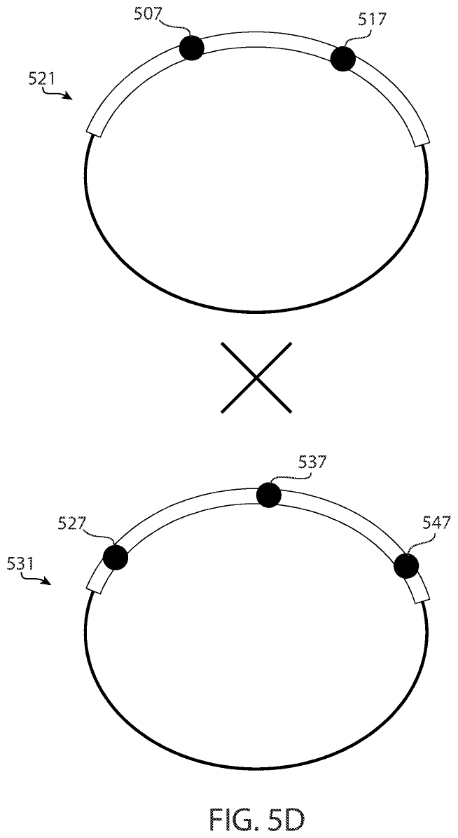

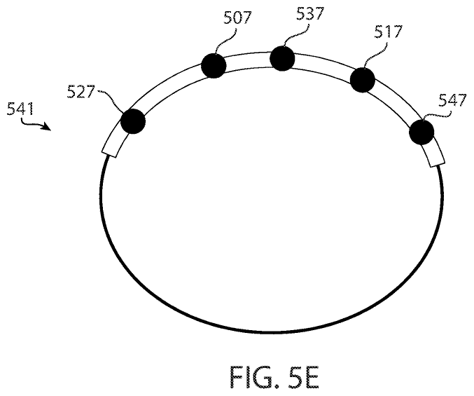

FIG. 5A presents an exemplary, schematic illustration of a non-limiting embodiment, where first plurality of primers 505 are complementary to the adjacent portion of nucleic acid 501, except at codons 507 indicated by black rectangles. In FIG. 5A, second plurality of primers 515 is complementary to nucleic acid 501 except at codons 517 indicated by black rectangles. Although the pluralities 505 and 515 are represented as including only five primers, this this number was chosen arbitrarily and each primer may, in practice, include any of a variety of appropriate numbers of primers, as discussed above.

In some embodiments, the first portion of the common template nucleic acid and the second portion of the common template nucleic acid do not overlap. For example, referring again to FIG. 5A, first plurality of primers 505 does not complement the same portion of nucleic acid 501 as second plurality of primers 515. However, in some embodiments the first portion of the common template nucleic acid and the second portion of the common template nucleic acid may at least partially overlap, as the disclosure is not so limited. Within each plurality of primers, at least some (e.g., all) of the primers may be non-complementary to the common template nucleic acid at exactly one codon. For example, referring to FIG. 5A, each primer of plurality of primers 505 is non-complementary to nucleic acid 501 only at codon 507 and plurality of primers 515 is non-complementary to nucleic acid 501 only at codon 517.

Upon amplification, the first plurality of primers can be used to produce a mutation at a first sequential position of a common template nucleic acid and the second plurality of primers can be used to produce a mutation at a second sequential position of the common template nucleic acid, different from the first sequential position. Thus, two pluralities of primers may be used to produce a library including every pairwise combination of the first mutation and the second mutation, producing a library including up to 20n×20m distinct nucleic acid sequences, where “n” is the length of the primers of the first plurality of primers, and “m” is the length of the second plurality of primers. The lengths may be independent of each other. In this way, a library of nucleic acids may be designed that differs from a common template nucleic acid at up to 1, up to 2, up to 3, up to 4, up to 5, up to 6, up to 7, up to 8, up to 9, up to 10, up to 15, up to 20, up to 25, up to 30, up to 35, up to 40, up to 45, up to 50, up to 60, up to 70, up to 80, up to 90, up to 100, up to 150, up to 200, up to 250, up to 300, up to 400, up to 500, up to 1,000 or more codons, by using an appropriate number of primers differing at exactly one codon from the template nucleic acid.

In some embodiments, separate pools of primers may be used to mutate different portions of the common template nucleic acid. For example, FIG. 5A may represent the contents of a first pool, including common template nucleic acid 501, first plurality of primers 505, and second plurality of primers 515, while FIG. 5B represents a second pool, including the common template nucleic acid 501, third plurality 525 of primers complementary to nucleic acid 501 except at one of codons 527, fourth plurality 535 of primers complementary to nucleic acid 501 except at one of codons 537, and fifth plurality 545 of primers complementary to nucleic acid 501 except at one of codons 547. In some embodiments, techniques such as the above example may produce a plurality of nucleic acids in a first pool, each differing from the common template nucleic acid at at least one codon, and a plurality of the nucleic acids in the first pool, each differing from the common template nucleic acid at at least one codon in a second pool. Each of these pools would contain a plurality of nucleic acids differing from the common template nucleic acid by exactly one codon. The use of different pools may be advantageous in some embodiments, since it may permit point mutations of nearby codons without using overlapping primers in the same pool.

As discussed above, one or more nucleic acids of a plurality of nucleic acids prepared using a first plurality of common template primers may be mutated using a second plurality of primers also present in the solution. For example, FIG. 5C presents a nucleic acid 551, representative of a plurality of nucleic acids formed by amplification of nucleic acid 501 with first plurality of primers 505 as shown in FIG. 5A. Codons 507, mutated by plurality of primers 505 shown in FIG. 5A, are represented with a black circle representing any arbitrary mutation that could result from first plurality of primers 505 shown in FIG. 5A. Nucleic acids 551 of the plurality of nucleic acids can then react with primers of the second plurality of primers 515, where each of the primers of second plurality of primers 515 differs from a second common template primer at exactly one codon. Second plurality of primers 515 can introduce a second mutation corresponding to the second portion of the common template nucleic acid, producing nucleic acids differing from the common template nucleic acid by exactly two codons.

Although these figures only directly pertain to pluralities of primers that differ from the common template primer at exactly one codon, it should, of course, be understood that other embodiments are also possible. For example, in some embodiments a plurality of primers includes primers that differ from the common template primer at two codons, depending on the embodiments. The use of primers differing at more than two codons may be used, in some embodiments, to increase the mutation rate.

In some embodiments, a first plurality of nucleic acids and a second plurality of nucleic acids may be used to synthesize a third plurality of nucleic acids, differing from the common template primer at more than one codon. For example, in some embodiments the first plurality of nucleic acids and the second plurality of nucleic acids may be mixed and shuffled using any of a variety of methods known to those of ordinary skill in the art. The mixing and amplifying may be used to produce at third plurality of nucleic acids, wherein the third plurality of nucleic acids is identical to the common template nucleic acid except where the first plurality of nucleic acids or the second plurality of nucleic acids differs from the common template nucleic acid. For example, the third plurality of nucleic acids may be produced using any of a variety of suitable techniques, such as nucleic acid shuffling (e.g., DNA shuffling), overlap amplification (e.g., overlap PCR), or staggered extension (e.g., a process comprising or consisting of priming a nucleic acid of the plurality, followed by repeated cycles of denaturation and very short annealing and polymerase-catalyzed extension, wherein denaturation allows the partially-extended chains to melt away from their initial nucleic acids and bind to other nucleic acids, prior to a subsequent extension step).

In some embodiments, the third plurality of nucleic acids is the library of nucleic acids. It should, of course, be understood that one or more additional steps of using an additional plurality of primers to synthesize an additional plurality of nucleic acids and/or of performing additional techniques such as DNA shuffling, overlap amplification, or staggered extension may also be performed, and that the steps of synthesis may be performed in any of a variety of appropriate orders, depending on the embodiment. The use of additional steps may contribute to the formation of even larger libraries.

FIGS. 5D-5E present a non-limiting example of formation of a third plurality of nucleic acids. In FIG. 5D, a first plurality of nucleic acids 521, which would be formed by the pool of FIG. 5A, and a second plurality of nucleic acids 531, which would be formed by the pool of FIG. 5E, are pooled together. Only one nucleic acid is shown for each plurality, but it represents a generic structure of nucleic acids of each plurality of nucleic acids. Codons 507, 517, 527, 537, and 547, mutated by pluralities 505, 515, 525, 535, and 545 of primers shown in FIGS. 5A-5B, are represented with black circles, each representing any arbitrary mutation that could result from the associated pluralities of primers shown in FIGS. 5A-5B. FIG. 5E represents the plurality nucleic acids 541 that would result from mixing and amplifying the pluralities of nucleic acids 521 and 531 shown in FIG. 5D. Nucleic acids 541 are identical to the common template nucleic acid, except where the nucleic acids 521 or nucleic acids 531 differ from the common template nucleic acid 501 shown in FIGS. 5A-5B.

Libraries (e.g., third pluralities of nucleic acids) provided herein may be of any of a variety of appropriate sizes. In some embodiments, a library of nucleic acid sequences produced by a method provided herein comprises greater than or equal to 10⁵, greater than or equal to 10⁶, greater than or equal to 10⁷, greater than or equal to 10⁸, greater than or equal to 10⁹, greater than or equal to 10¹⁰, greater than or equal to 10¹¹, greater than or equal to 10¹², greater than or equal to 10¹³ or more sequences. According to some embodiments, a library of nucleic acid sequences produced by a method provided herein comprises less than or equal to 10¹⁴, less than or equal to 10¹³, less than or equal to 10¹², less than or equal to 10¹¹, less than or equal to 10¹⁰, less than or equal to 10⁹, less than or equal to 10⁸, or less sequences. Combinations of these ranges are also possible. For example, in some embodiments a library of nucleic acids comprises greater than or equal to 10⁵ and less than or equal to 10¹⁴ sequences.

The methods provided herein may provide any of a variety of suitable rates of mutations per round. Notably, high mutation rates per round may be advantageous for preparing libraries as described herein, since high mutation rates may allow the preparation of larger nucleotide libraries with fewer rounds of mutation. In some embodiments, a method provided herein includes a step of synthesizing a plurality of nucleic acids with a maximum mutation rate less than or equal to 32 mutations per kb per round, less than or equal to 30 mutations per kb per round, less than or equal to 25 mutations per kb per round, less than or equal to 22 mutations per kb per round, less than or equal to 20 mutations per kb per round, less than or equal to 15 mutations per kb per round, less than or equal to 12 mutations per kb per round, less than or equal to 10 mutations per kb per round, less than or equal to 5 mutations per kb per round, less than or equal to 2 mutations per kb per round, or less. In some embodiments, a method provided herein includes a step of synthesizing a plurality of nucleic acids with a maximum mutation rate of greater than or equal to 1 mutations per kb per round, greater than or equal to 2 mutations per kb per round, greater than or equal to 5 mutations per kb per round, greater than or equal to 10 mutations per kb per round, greater than or equal to 12 mutations per kb per round, greater than or equal to 15 mutations per kb per round, greater than or equal to 20 mutations per kb per round, greater than or equal to 22 mutations per kb per round, greater than or equal to 25 mutations per kb per round, greater than or equal to 30 mutations per kb per round, or greater. Combinations of these ranges are also possible. For example, in some embodiments, a method provided herein includes a step of synthesizing a plurality of nucleic acids with a maximum mutation rate of greater than or equal to 1 mutations per kb per round and less than or equal to 32 mutations per kb per round.

Another advantage of methods provided herein is that mutagenesis performed using a plurality of primers provided herein may have a very low or negligible likelihood of causing a frame-shift during mutagenesis. This may result in the preparation of libraries with relatively few early stop-codons, even when libraries are prepared using a high mutation rate as discussed above. Libraries (e.g., third pluralities of nucleic acids) provided herein include a very small proportion of sequences with early stop codons. In some embodiments, less than or equal to 10%, less than or equal to 5%, less than or equal to 2.5%, less than or equal to 1% or less of the sequences of the library comprise an early stop codon. According to some embodiments, greater than or equal to 0.1%, greater than or equal to 0.2%, greater than or equal to 0.5% or greater than or equal to 1% of the sequences of the library comprise early stop codons. Combinations of these ranges are also possible. For example, in some embodiments a library of nucleic acids produced according to a method provided herein includes early stop codons in greater than or equal to 0.1% and less than or equal to 10 wt %. of its sequences.

In some embodiments, sequential amplification steps may be used to produce a library. For example, a first plurality of nucleic acids may be synthesized, e.g., as described above. Then, at least some nucleic acids of the first plurality of nucleic acids may be amplified using a second plurality of primers to synthesize a second plurality of nucleic acids, e.g., as described above.

In some embodiments, certain systems and methods comprise determining a subset of the first plurality of nucleic acids that is associated with favorable mutations of the common template nucleic acid. Such a favorable subset may be identified by any of a variety of appropriate methods. For example, the subset may be identified by selecting for nucleic acids that bond to a target substrate. As another example, in some embodiments each of the nucleic acids are used to express a protein, and the activity of that protein is measured by an appropriate method (e.g., by detecting activity of a probe, such as a change in fluorescence of a fluorescent probe, or a color change of a colorometric indicator). For example, the protein may be mixed with an enzyme substrate, and enzymatic activity of the protein may be detected, as described in the examples below. The protein may be expressed by any of a variety of appropriate methods. For example, the protein may be expressed by performing a translation reaction to synthesize it, or by expressing the protein in a plasmid by inserting the nucleic acid into the plasmid. Other techniques are also possible, and the disclosure is not so limited.

Generally, any of a variety of appropriate numbers of nucleic acids of the first plurality of nucleic acids may be used to synthesize the second plurality of nucleic acids. For example, in some embodiments, greater than or equal to 1, greater than or equal to 2, greater than or equal to 5, greater than or equal to 10, greater than or equal to 20, greater than or equal to 50, or more nucleic acids of the first plurality may be used to synthesize the second plurality of nucleic acids.

In some embodiments, the less than or equal to 100%, less than or equal to 99%, less than or equal to 95%, less than or equal to 90%, less than or equal to 75%, less than or equal to 50%, or less of the nucleic acids of the first plurality may be used to synthesize the second plurality of nucleic acids. Combinations of the forgoing ranges are possible. For example, in some embodiments, at least one nucleic acid of the first plurality and less than or equal to 100% of the nucleic acids of the first plurality of nucleic acids may be used to synthesize the second plurality of nucleic acids. Other ranges are also possible. The nucleic acids of the first plurality used to synthesize the second plurality of nucleic acids may be collected by any of a variety of suitable methods. For example, the first plurality of nucleic acids may be broken into droplets, as discussed in greater detail below.

One advantage of sequential mutation is that the determination of the favorable subset may be performed between amplification steps, and a subset of a first plurality of nucleic acids that is associated with favorable mutations of the common template nucleic acid may be selected for amplification. For example, in some embodiments, a most favorable nucleic acid from the first plurality can be used to synthesize the second plurality of nucleic acids using the second plurality of primers. In some embodiments, the most favorable nucleic acid can be treated as a second common template nucleic acid, and primers may be designed to be complementary to the mutated codon, while introducing new mutations at the site of a different codon.

In some embodiments, some or all nucleic acids of the first plurality of nucleic acids are used to form the second plurality of nucleic acids. For example, the nucleic acids of the first plurality may be included in a single mixture, and may be amplified together within that mixture using the second plurality of primers. However, in some embodiments, the first plurality of nucleic acids may be partitioned, e.g., by breaking a mixture comprising the nucleic acids into a plurality of droplets and amplifying nucleic acids therein, as discussed below. Then, the second plurality of primers may be introduced into at least some of the plurality of droplets, in order to synthesize the second plurality of nucleic acids within the droplets.

Synthesis of the nucleic acids described herein may comprise an entirely sequential process of mutations, where one codon is mutated at a time. In some embodiments, the nucleic acids, one or more step may be used to introduce multiple mutations during the same reaction, as discussed above. Combinations of these steps may also be used. For example, in some embodiments, a method may comprise a first step, where 5 pluralities of primers are used to prepare a first plurality of nucleic acids, each differing by up to 5 codons from a common template nucleic acid. (Other numbers of primers may be used in other embodiments, for example, 2, 3, or 4, or 6, 7, 8, 9, 10, etc.; 5 is used by way of example only.) This method may further comprise using a sixth plurality of primers to introduce a mutation in exactly one additional codon of at least some nucleic acids of the first plurality. Intermediate steps, such as identifying a favorable subset of the first plurality of nucleic acids, may be used in concert with such a method. Such a method may be useful for identifying useful mutations of proteins that are already favorably mutated relative to a wild type protein, permitting a directed process of library evolution.

Furthermore, a patent application filed on Sep. 26, 2022, entitled “Methods and Systems for Full Gene Length Single Point Mutagenesis,” U.S. Provisional Patent Application Ser. No. 63/410,116, by Weitz et al., is also incorporated herein by reference in its entirety. Also, U.S. Provisional Patent Application Ser. No. 63/410,140, filed Sep. 26, 2022, entitled “Systems and Methods for Screening of Large Gene Libraries,” by Weitz, et al., is also incorporated herein by reference in its entirety.

In some aspects, nucleic acids and proteins identified by one or more of the methods provided herein are provided. According to some embodiments, a protein provided herein is a mutant of the Lipase A (“LipA”) enzyme. For example, in some embodiments, a protein provided herein comprises a sequence that is at least 70%, at least 80%, at least 90%, or at least 95% identical to one of Seq. ID. Nos. 2-24 and is Seq. ID. No. 25. In some embodiments, a protein provided herein comprises one of Seq. ID. Nos. 2-24]. For example, a protein provided herein may be one of Seq. ID. Nos. 2-24.

In some embodiments, a nucleic acid provided herein is a nucleic acid that encodes a mutant of the LipA enzyme. For example, in some embodiments a nucleic acid provided herein comprises a sequence that is at least 70%, at least 80%, at least 90%, or at least 95% identical to one of Seq. ID. Nos. 26-48 and is not Seq. ID. No. 49. In some embodiments, a nucleic provided herein comprises one of Seq. ID. Nos. 26-48. For example, a protein provided herein may be one of Seq. ID. Nos. 26-48.

The following examples are intended to illustrate certain embodiments of the present disclosure, but do not exemplify the full scope of the disclosure.

### Example 1

This example demonstrates that detection of an active nucleic acid in a droplet comprising a library of 100,000 distinct nucleic acid sequences is viable using a non-limiting example of a method described herein. The active nucleic acid sequence is able to express an active protein (wild-type Lipase A) that is capable of activating a target substrate (resorufin acetate) and was deliberately added to the library of at least 100,000 inactive nucleic acid sequences, to determine whether it could be identified. In this example, a plurality of droplets was prepared, of which one in every ten droplets included the wild-type Lipase A gene (the “WT-gene”). A positive control was also performed, wherein a control plurality of droplets was prepared that included only the WT-gene. In the control plurality of droplets, only one in every ten droplets included the WT-gene.

A gene library was prepared using a construct of the target nucleic acids and a pET28a+ vector. T7 specific primers were used for RNA transcription. A plurality of droplets was prepared using the gene library. The plurality of droplets had an average diameter of 30 microns, and included approximately 500,000 sequences.

For each initial plurality of droplets, the following steps were performed:
1. An in vitro transcription and translation (IVTT) system and resorufin-acetate was included in the initial 30 micron droplets. The droplets were incubated for 4 h to permit transcription and translation of wild-type Lipase A.
2. Droplet-based microfluidics was used to sort the droplets based on fluorescent activity resulting from the reaction of resorufin acetate with wild-type Lipase A. Droplets that demonstrated fluorescent activity were separated from the other droplets.
3. Nucleic acids present in the droplets demonstrating fluorescent activity were merged, and the nucleic acids therein were amplified using PCR and purified with a PCR clean-up kit.
4. The amplified nucleic acids were separated into a second plurality of droplets having a 15 micrometer diameter and including only 1,500 nucleic acid sequences.
5. Step 1 was repeated.
6. Step 2 was repeated.
7. Step 3 was repeated.
8. The amplified nucleic acids were separated into a third plurality of droplets having a 15 micrometer diameter and including only 5 nucleic acid sequences per droplet.
9. Step 1 was repeated.
10. Step 2 was repeated.
11. Step 3 was repeated.
12. The amplified nucleic acids were separated into a fourth plurality of droplets having a 15 micrometer diameter and including only 1 nucleic acid sequence per ten droplets (such that nine out of every ten droplets included no nucleic acids).
13. Step 1 was repeated.
14. Step 2 was repeated.
15. Nucleic acids in the active droplets of the fourth plurality were sequenced.

This method successfully isolated the WT-gene from the library, and allowed it to be identified, demonstrating the efficacy of this approach for library refinement.

### Example 2

This prophetic example demonstrates screening a nucleic acid library for improved thermal stability of a synthesized protein. In this example, a first plurality of droplets is prepared that includes a library of nucleic acids that expresses a variety of proteins. First, nucleic acids in the droplets are transcribed and translated using IVTT. Then, the droplets are thermally shocked (e.g., by holding the droplets at 70° C. for 30 minutes). Finally, an active substrate is pico-injected into the droplets, which are subsequently sorted for activity of the active substrate using microfluidics. Steps of breaking the droplets, amplifying the nucleic acids therein, and sorting the droplets based on activity of the active substrate are iterated to identify one or more acids from the library capable of producing proteins that resist the thermal shock. This prophetic example demonstrates the viability of the claimed methods for identifying favorably stable proteins based on droplet-based library screening.

### Example 3

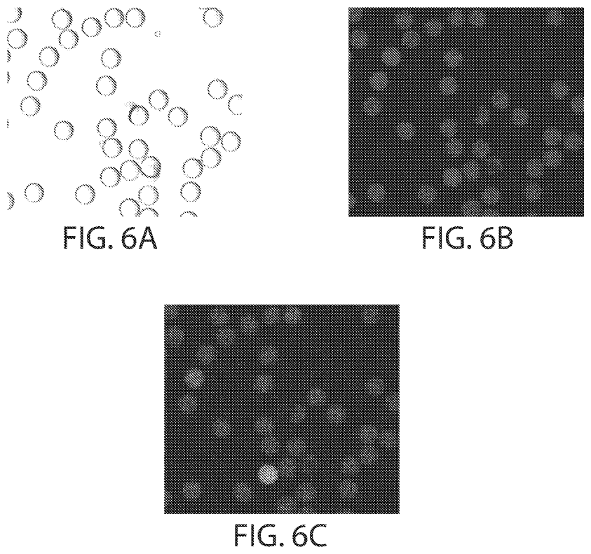

This example demonstrates that a nucleic acid library could be screened to identify variants that activate multiple target substrates. A plurality of droplets was prepared as in Example 1; however, in addition to resorufin acetate (RA), pyranine (HTPS) was used as a target substrate. Wild-type Lipase A did not activate HTPS. A library of 10¹² nucleic acids (“the LipA library”, included a plurality of about 2×10⁶ droplets, each including 500,000 nucleic acids) mutated from the WT-gene was prepared and screened for RA and HTPS activity. HTPS was a green fluorophore and RA was a red fluorophore, so activity of each fluorophore could be detected simultaneously. For example, FIGS. 6A-6C present confocal microscope images of the plurality of droplets by transmission (FIG. 6A) detection of red fluorescence (570-580 nm; FIG. 6B) and green fluorescence (450-510 nm; FIG. 6C). (Both figures are included in greyscale, but the relative brightness of various pixels indicates the relative fluorescent intensity within the specified range of wavelengths.) Droplets with high activity of both RA and HTPS, indicated by high fluorescent intensity in the red and green wavelengths, may be identified and sorted. This example demonstrates that a method such as that of Example 1 could be used to identify multifunctional proteins within a target library.

### Example 4

This example demonstrates that a nucleic acid library may be screened to identify variants that cleave a target peptide. The LipA library described in Example 3 was used along with the target substrate 5-FAM-PQPQLPYPQK-qxl (SEQ ID NO: 1), where 5-FAM was a fluorophore and qxl was a 5-FAM quencher. The peptide itself had no fluorescent activity, but once cleaved, the 5-FAM was able to fluoresce, producing a fluorescent signal. In order to identify variants able to cleave 5-FAM-PQPQLPYPQK-qxl (SEQ ID NO: 1), the following method was used.

The LipA library was prepared using a construct of the library nucleic acids and a pET28a+ vector. T7 specific primers were used for RNA transcription. A plurality of droplets was prepared using the LipA library. For the initial plurality of droplets, the following steps were performed:
1. An in vitro transcription and translation (IVTT) system and resorufin-acetate was included in the initial 30 micron droplets. The droplets were incubated for 4 h to permit transcription and translation of LipA library.
2. Droplet-based microfluidics was used to sort the droplets based on fluorescent activity resulting from the reaction of 5-FAM-PQPQLPYPQK-qxl (SEQ ID NO: 1) with translated proteins. Droplets that demonstrated fluorescent activity were separated from the other droplets.
3. Droplets demonstrating fluorescent activity were merged, and the nucleic acids therein were amplified using PCR, and purified with a PCR clean-up kit.
4. The nucleic acids were separated into a second plurality of droplets having a 15 micrometer diameter and including only 1,500 nucleic acid sequences.
5. Step 1 was repeated.
6. Step 2 was repeated.
7. Step 3 was repeated.
8. The nucleic acids were separated into a third plurality of droplets having a 15 micrometer diameter and including only 5 nucleic acid sequences per droplet.
9. Nucleic acids in the active droplets of the third plurality were sequenced.

Rather than identifying a single best variant, partial sequencing of the nucleic acids in the third plurality of droplets was used to identify a plurality of variants that were enriched in the third plurality of droplets. At least three mutations of wild-type Lipase A were shown to be enriched in the sequenced populations. The first variant (present in 5.53% of the nucleic acids in the third plurality of droplets) had the mutations K35R, V39A, and V96I. The second variant (present in 2.61% of the nucleic acids in the third plurality of droplets) had the mutation K35R, but did not include any other identified mutations. The third variant (present in 1.98% of the nucleic acids in the third plurality of droplets) included the mutations M8K, S16N, A81E, and N98S. These results demonstrate that library refinement may be used identify variants of interest in a library of nucleic acids, illustrating the utility of the methods described herein.

### Example 5

This prophetic example demonstrates that a method described herein can be used to screen a library to identify peptides that bind to a specific receptor. In this prophetic example, a DNA library is prepared that can express a library of peptides consisting of up to 10 amino acids. The library comprises DNA sequences that include, in order: 4-5 primer binding sites for nested PCR, a T7 promotor, a ribosome binding site, a START codon, a peptide library sequence, a STOP codon, a T7 terminator, and 4-5 primer binding sites.

Up to 10⁷ nucleic acids from the DNA library (which includes 10¹² nucleic acids) are encapsulated into each droplet of a first plurality of 10⁶ droplets that further comprise an in vitro transcription and translation mixture and a reporter. In vitro transcription and translation is performed for 4 h at 37° C., and droplets are sorted based on a fluorescence increase from the reporter cell. Sorted droplets with high fluorescence are merged and nucleic acids therein are amplified using the primer binding sites, and purified using a PCR clean-up kit. Then, the nucleic acids present in the merged droplets of the first plurality are separated into a second plurality of droplets, including 1,000 droplets with around 10⁴ nucleic acids per droplet. Next, IVTT is performed again and the droplets of the second plurality of droplets are sorted based on fluorescent activity, and the high fluorescence droplets are merged, and amplified. The nucleic acids present in the merged droplets of the second plurality are separated into a third plurality of 1000 droplets, including about 10 nucleic acids per droplet. Next, the droplets of the third plurality of droplets are sorted based on fluorescent activity, and the high fluorescence droplets are merged, and amplified. The amplified nucleic acids are then separated into a fourth plurality of droplets, including, on average, one nucleic acid per droplet. The nucleic acids of the fourth plurality of droplets are then sequenced to identify active nucleic acids.

This description demonstrates how some of the methods described herein may be used to identify nucleic acids expressing active peptides that interact with a reporter cell.

### Example 6

This example describes the preparation and screening of the LipA library initially described in Example 1. Wild-type LipA (“WT-LipA”), originating from B. Subtilis, was chosen as a common template nucleic acid for preparation of the LipA library. Eighteen pluralities of primers each, covering 10 codons were ordered to cover the full WT-LipA gene. Each plurality of primers included 10×19 primers. Each primer of a plurality was non-complementary to exactly one codon of the corresponding portion of the common template nucleic acid, and could thus be used to synthesize a nucleic acid differing from the common template nucleic acid at exactly one codon. Thus, each plurality of primers could be used to synthesize a corresponding plurality of up to 190 nucleic acids—the number of nucleic acids necessary to encode every esterase mutant including a single amino acid mutation in the portion of the esterase encoded by the portion of the common template nucleic acid corresponding to the primers. Primers were ordered through Integrated DNA Technology (IDT). Mutations of the first and last 5 codons were excluded. Pluralities of primers were designed to fully overlap in mutated regions, and 9, 6, or 3 nucleotides complementary to the wild-type sequence were added on the 5′ end when mutating the first, second, or third amino acid in that region, respectively, to ensure proper binding of every primer. The same was done at the 3′ end when mutating the last three amino acids. All pluralities of primers were ordered in a final concentration of 50 pmols per primer and dissolved in IDTE buffer (10 mM Tris pH 8.0, 0.1 mM EDTA) to a final concentration of 100 μM, and were diluted with nuclease-free water to a reaction-ready concentration of 10 μM. PCR was used to prepare pluralities of nucleic acids using the pluralities of primers under the following heating conditions: 95° C. for 5 minutes; 95° C. for 20 seconds, 55° C. for 30 seconds, 65° C. for 3 minutes—30×; 65° C. for 5 minutes. When PCR was done, 1 μL of DpnI was added and the mixture was incubated at 37° C. for 1 hour. Two pools of primers were prepared after PCR. Pool 1 contained pluralities 1, 3, 5, 7, 9, 11, 13, 15, and 17, and pool 2 contained pluralities 2, 4, 6, 8, 10, 12, 14, 16, and 18.

Mutations were introduced into a wild-type sequence using Quick Change Lighting Multi Site Directed Mutagenesis kit (Agilent, CA, US) for each pool. For second strand synthesis, 10 μL of each pool was transformed into a 100 μL aliquot of E. coli Turbo cells (NEB, MA, US). Transformation was done according to the instruction manual and cultured overnight in 10 mL of LB-media supplemented with 50 μg/mL kanamycin at 37° C. Only 50 μL out of 10 mL was plated on a solid plate, to estimate library size. Plasmids were extracted using Plasmid Isolation Kit (NEB, MA, US).

Plasmids extracted from liquid culture were used for preparation of the LipA library. After the site-specific mutagenesis reactions and plasmid extractions, an additional layer of variability was introduced by executing a round of DNA shuffling. The pluralities of nucleic acids were shuffled using the staggered extension process (“StEP”) by mixing 2.5 μL of the pluralities of LipA mutants (1 ng/μL), 1.5 μL of 10 μM T7 forward primer, 1.5 μL of 10 μM T7 reverse primer, 50 μL of 2×Taq polymerase master mix (NEB, MA, US) and 44.5 μL of nuclease-free water. PCR was done under the following conditions: 94° C. for 30 seconds, 55° C. for 5 seconds—99×. PCR products were purified using PCR Clean up Kit (NEB, MA, US) and were for the second reaction: 5 μL of PCR product (5 ng/μL), 15 μL of 10 μM T7 forward primer, 15 μL of 10 μM T7 reverse primer, 500 μL of 2× Taq polymerase master mix, and 465 μL of nuclease-free water. The mixture was split into 20 PCR tubes and PCR was done under the same conditions. All 20 PCR reactions were purified separately and used as template DNA for 20 new reactions: 2.5 μL of template DNA (1 ng/μL), 1.5 μL of 10 μM T7 forward primer, 1.5 μL of 10 μM T7 reverse primer, 50 μL of 2×Taq polymerase master mix (NEB, MA, US), and 44.5 μL of nuclease-free water. After PCR, all samples were mixed and purified using the same kit to produce the LipA library (which totaled around 10¹² nucleic acids).

### Example 7

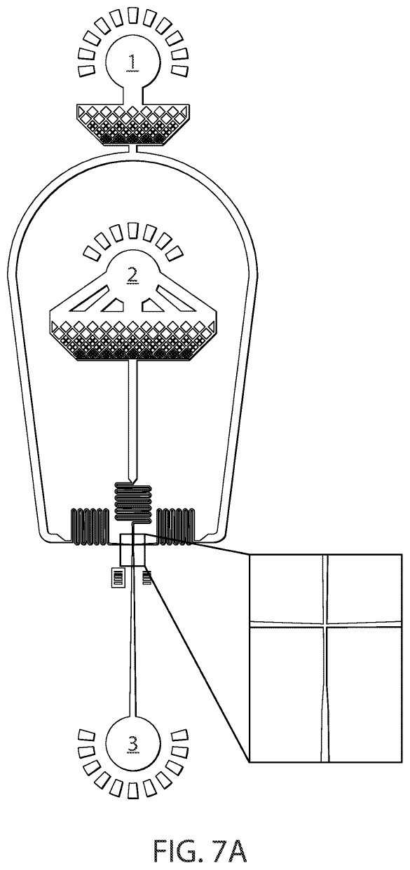

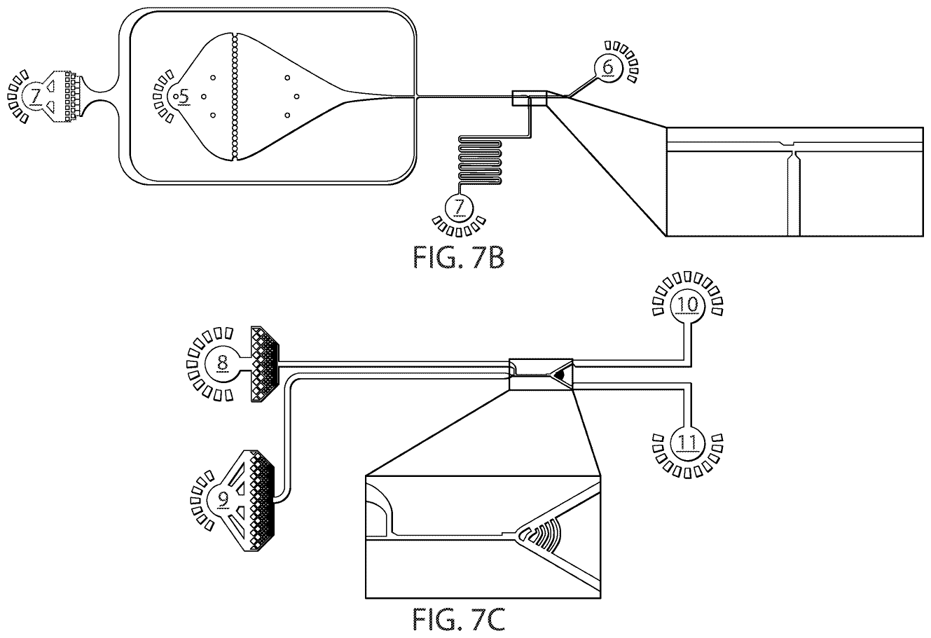

This example demonstrates high throughput screening of the LipA library to identify variants with high thermal stability. A microfluidic device was designed in order to process pluralities of droplets comprising LipA library sequences. The microfluidic device had the design shown in FIGS. 7A-7C. FIG. 7A shows the design of the devices used to encapsulate and process the LipA library genes and the in vitro protein synthesis reagents. Labels 1 to 3 connote oil inlet 1, aqueous fluid inlet 2, and collection outlet 3. FIG. 7B shows the design of the device used to pico-inject substrate into each water-in-oil droplet. Labels 4-7 connote spacing oil inlet 4, droplet reinjection inlet 5, substrate inlet 6, and collection outlet 7. FIG. 7C shows the design of the device used to screen the droplets based on fluorescent intensities. Labels 8-11 connote spacing oil inlet 8, droplet reinjection inlet 9, sorted droplet collection outlet 10, and waste outlet 11. The devices (droplet generator, pico-injector, and droplet sorter) of FIGS. 7A-7C) were designed utilizing AutoCAD and were produced as photomasks (CAD/Art Services, Inc.). The devices were fabricated through the well-established techniques of soft lithography, employing SU8-on-Silicon-wafer masters and PDMS-on-glass devices. Polydimethylsiloxane (PDMS) (Sylgard 184) was poured onto the masters, and the masters were baked at 65° C. overnight to cure the PDMS. Subsequently, each PDMS device was cautiously peeled from the master and sealed to a pristine glass slide (Corning, 2947). For devices incorporating electrodes, the electrodes were integrated into the design as channels within the microfluidic devices. These channels were filled with a low melting point metal alloy (Indalloy 19, 0.020 in. diameter) while the devices were heated on a hot plate. Terminal blocks were added to the punched holes in the devices to facilitate electrical connections during droplet processing. Following the fabrication process, Aquapel (fluoroalkylsilanes) was injected through the punched holes of the devices and pressurized air was used to impel it through the microfluidic channel walls to render hydrophobic internal surfaces of the channels. Excess Aquapel was expelled using compressed air, and the device was baked at 65° C. overnight.

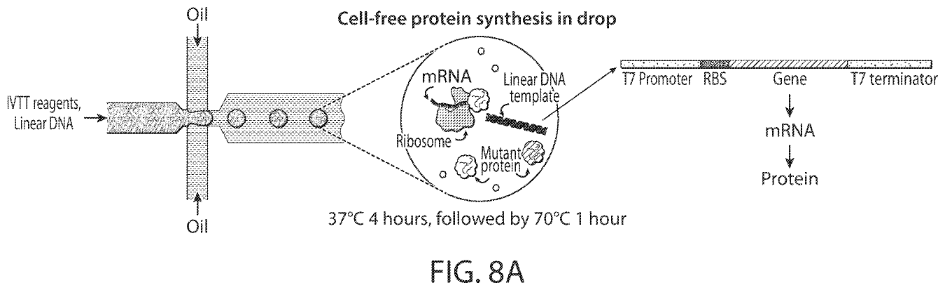

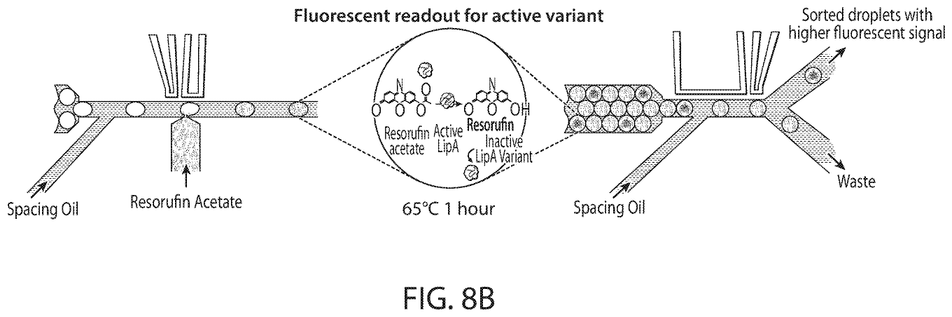

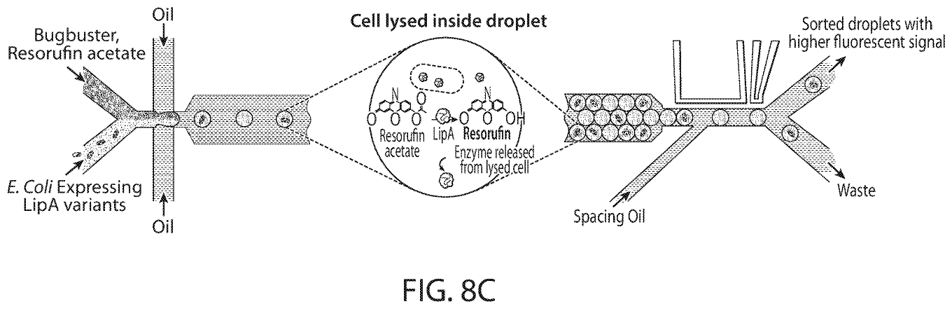

Proteins were synthesized by multiplex cell-free ultra-highthroughput enzyme evolution as shown schematically in FIGS. 8A-8C using the devices of FIGS. 7A-7C. FIG. 8A provides a non-limiting, schematic illustration of cell-free protein synthesis in drop. FIG. 8B provides a non-limiting, schematic illustration of pico-injection of substrate, followed by fluorescence-based droplet sorting. FIG. 8C shows schematics of co-encapsulation of one bacteria cell host expressing enzyme mutants, fluorescent substrate, and lysis buffer, followed by fluorescence-based droplet sorting.

The concentration of mutant genes per unit volume was precisely quantified, and the concentration of the previously established linear DNA gene library was diluted accordingly. A known quantity of linear genes was then encapsulated, together with in vitro Protein Synthesis reagents (PURExpress, New England Biolabs), within water-in-oil microdroplets using the device shown in FIG. 7A. The linear genes were combined with the in vitro protein synthesis reagents and loaded into a 1 mL syringe (Norm-Ject® Sterile Luer-Slip Syringes). This mixture was delivered to the droplet generator's aqueous inlet channel via a syringe needle attached to polyethylene tubing (BB31695-PE/2, Scientific Commodities, Inc.). Concurrently, a surfactant solution containing 2% (w/v) fluorinated surfactant (RAN Biotechnologies, 008-FluoroSurfactant) in HFE 7500 (3M) was loaded into a 3 mL syringe (BD Luer-Lok 3-mL syringe) and introduced to the oil inlet channel of the droplet generator using a similar setup. Harvard Apparatus Pumps were employed to exert controlled positive pressure on the syringes, yielding flow rates of 60 L/h for the aqueous mixture and 300 μL/h for the oil-surfactant solution, resulting in the generation of monodisperse water-in-oil emulsions.

The emulsions were collected in an Eppendorf tube, over which 100 μL of mineral oil (MI499, Spectrum Chemical MFG Corp.) was carefully layered to prevent evaporation. The tube was incubated in a thermostatic heat block at 37° C. for 4 hours to facilitate in-drop protein synthesis. Subsequently, the samples were subjected to heat shock at 70° C. for one hour to inactivate any non-thermostable variants.

After incubation, all the linear genes were assumed to be transcribed and translated into proteins of LipA mutants. The droplets were re-injected into a picoinjector device for pico-injection of resorufin acetate substrate. Resorufin acetate, was dissolved in DMSO (7.5 M) to make stocks and 30 μL of resorufin acetate stock was diluted in 1 mL of filtered PBS, which was then loaded into a 1 mL syringe. This mixture was then delivered to the picoinjector inlet via a Flow EZ™ flow control system (Fluigent Inc.). After loading the droplets, HFE-7500 oil, and the substrate solution into the device via the method as described before, the pressure was meticulously calibrated within the injector channel to minimize any dripping. The droplets were close-packed and periodically introduced to the main flow channel by modulating the flow rates of the oil and droplets. Upon stabilization, a voltage was applied across electrodes, effecting the pico-injection of the substrate solution into each droplet at an approximate rate of 5 kHz. The droplets were then collected into an Eppendorf tube placed in a thermostatic heat block setting at 65° C. for the reaction with resorufin acetate to complete. A layer of 100 μL of mineral oil (MI499, Spectrum Chemical MFG Corp.) was carefully layered on top of the emulsion to prevent evaporation.

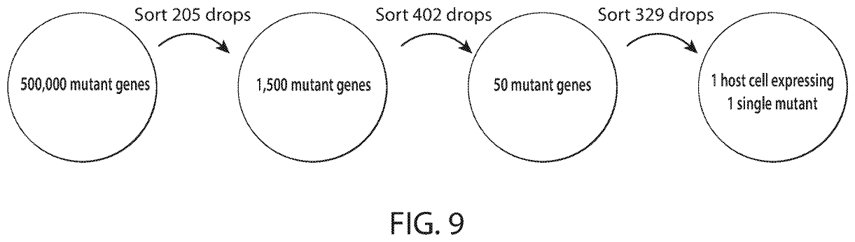

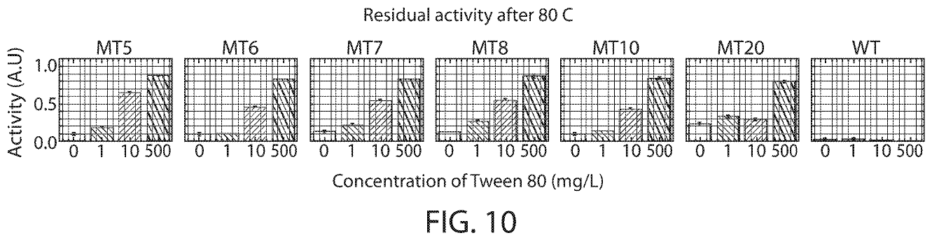

Four rounds of multiplexed sorting were performed. The full LipA library of approximately 10¹² LipA mutants was screened by screening 2,103,839 droplets, each comprising an average of 500,000 mutants, of which 205 droplets were determined to be activity droplets and were sorted for subsequent processing, as indicated schematically in FIG. 9.

To determine the activity droplets, a 532 nm excitation laser was placed on the entrance of the sorter. If the fluorescent reaction product in one droplet was higher than the set fluorescence threshold, it was pulled by the electric field into an adjacent channel, which was then collected through polyethylene micro-tubing to an Eppendorf tube placed on ice. The chip operated at 600-1000 drops·s⁻¹, probing ~3·10⁶ cells·h⁻¹. By applying electric fields of 50 V at a frequency of 25 kHz, more than 2 million droplets were screened, and 205 droplets were sorted (205×500,000=100,250,000 genes). The emulsion was broken to forma second plurality of droplets by adding 200 μL of 20% (v/v) PFO (1H,1H,2H,2H-Perfluoro-1-octanol, 370533 Sigma Aldrich) in HFE 7500 (3M), and the aqueous phase was purified using a DNA clean-up kit (Monarch® PCR & DNA Cleanup Kit, New England Biolabs). The DNA was eluted in 5 μL of ddH2O. Using nested PCR primers, the sorted genes were amplified with a high-fidelity polymerase (NEB Q5® DNA Polymerase, New England Biolabs) following a standard thermocycling protocol.

In the second round, the second plurality of droplets, including 808,052 droplets, each comprising an average of approximately 1,500 LipA mutants as indicated schematically in FIG. 9. The second plurality of droplets was encapsulated with the in vitro Protein Synthesis reagents into 30 m water-in-oil droplets. The emulsion was incubated at 37° C. for 4 hours for in-drop protein synthesis, followed by a heat shock at 70° C. for 1 hour to inactivate the non-thermostable variants, RA picoinjection, and the second plurality of droplets was collected in mineral oil. As indicated in FIG. 9, 402 activity droplets were identified.

In the third round, a third plurality of 694,169 droplets was prepared, each comprising approximately 50 LipA mutants as indicated schematically in FIG. 9. The sequences of the third plurality of plurality of droplets were amplified and screened to identify 329 activity droplets comprising an average of 1 LipA mutant per droplet, as indicated schematically by FIG. 9.

Finally, the fourth plurality of droplets was broken to form a fifth plurality of 3,513,257 droplets, which were amplified and sorted as previously described to ensure a high likelihood that each resulting activity droplet included exactly 1 LipA mutant. The DNA plasmids were purified, and the plasmids were transformed into high-efficiency competent cells (Turbo Competent E. coli, New England Biolabs) for plasmid replication and extraction. The sorted plasmids were linearized using a restriction enzyme (NruI-HF, NEB #R3192S) and sequenced using PacBio Sequel II by commercial providers (Icahn Institute for Genomics and Multiscale Biology) to identify the active, thermostable sequences provided in Table 1. The corresponding nucleic acid sequences are provided in Table 2.

TABLE 1
Amino acid sequences identified in final plurality of activity droplets.

| Mutant | | |
|---|---|---|
| name | Seq. Id. No. | Amino Acid Sequence |
| MT1 | 2 | MEHNPVVMVHGIGGAGFNFAGIKSYLVSQGWSRDKLHA |
| | | VDFWDKTGTNYNNGPVLSRFVQKVLDETGAKKVDIVAH |
| | | SMGGANTLYYIKNLDGGNKVANVVTLGGANRLTTGKAP |
| | | PGTDPNQKILYTSIYSSADEKVSNDLSRLDGARNVQIHGV |
| | | GHIGLLYSSQVNSLIKEGLNGGGQNTN |
| MT2 | 3 | MEHNPVVMVHGIGGASFNFAGIKSYLVSQGWSRDKLYAV |
| | | DFWDKTGTNYNNGPVLSRFVQKVLDETGAKKVDIVAHS |
| | | MGGANTLYYIKNLDGGNKVANVVTLGGANRLTTGKALP |
| | | GTDPNQKILYTSIYSSADPIVANSLSRLDGARNVQIHGVGH |
| | | IGLLYSSQVNSLIKEGLNGGGQNTN |
| MT3 | 4 | MEHNPVVMVHGIGGAGFNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADQDVINCLSRLDGARNVQIHGVG |
| | | HIGLLYSSQVNSLIKEGLNGGGQNTN |
| MT4 | 5 | MEHNPVVMVHGIGGAGKNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADRDVGNYLSRLDGARNVQIHGV |
| | | GHIDLLYSSQVNSLIKEGLTGGGQNTN |
| MT5 | 6 | MEHNPVVMVHGIGGASTNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADEEVDNDLSRLDGARNVQIHGV |
| | | GHIDLLYSSQVNSLIKEGLSGGGQNTN |
| MT6 | 7 | MEHNPVVMVHGIGGASKNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADEEVDNDLSRLDGARNVQIHGV |
| | | GHIGLLYSSQVNSLIKEGLNGGGQNTN |
| MT7 | 8 | MEHNPVVMVHGIGGASENFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADEEVDNDLSQLDGARNVQIHGV |
| | | GHIDLLYSSQVNSLIKEGLDGGGQNTN |
| MT8 | 9 | MEHNPVVMVHGIGGASANFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADEEVDNDLSRLDGARNVQIHGV |
| | | GHIDLLYSSQVNSLIKEGLSGGGQNTN |
| MT9 | 10 | MEHNPVVMVHGIGGASWNFAGIKSYLVSQGWSLDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADEEVDNDLSRLDGARNVQIHGV |
| | | GHIGLLYSPQVNSLIKEGLQGGGQNTN |
| MT10 | 11 | MEHNPVVMVHGIGGASNNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADEEVDNDLSRLDGARNVQIHGV |
| | | GHIGLLYSSQVNSLIKEGLNGGGQNTN |
| MT11 | 12 | MEHNPVVMVHGIGGASSNFAGIKSYLVSQGWSGDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADEEVDNDLSRLDGARNVQIHGV |
| | | GHIGLLYSSQVNSLIKEGLNGGGQNTN |
| MT12 | 13 | MEHNPVVMVHGIGGASSNFAGIKSYLVSQGWSRDKLYAV |
| | | DFWDKTGTNYNNGPVLSRFVQKVLDETGAKKVDIVAHS |
| | | MGGANTLYYIKNLDGGNKVANVVTLGGANRLTTGKALP |
| | | GTDPNQKILYTSIYSSADEEVDNDLSRLDGARNVQIHGVG |
| | | HIGLLYSSQVNSLIKEGLDGGGQNTN |
| MT13 | 14 | MEHNPVVMVHGIGGASFNFAGIKSYLVSQGWSRDKLYAV |
| | | DFWDKTGTNYNNGPVLSRFVQKVLDETGAKKVDIVAHS |
| | | MGGANTLYYIKNLDGGNKVANVVTLGGANRLTTGKALP |
| | | GTDPNQKILYTSIYSSADEEVDNDLSRLDGARNVQIHGVG |
| | | HIGLLYSSQVNSLIKEGLNGGGQNTN |
| MT14 | 15 | MEHNPVVMVHGIGGASKNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADNDVDNDLSRLDGARNVQIHGV |
| | | GHIGLLYSSQVNSLIKEGLSGGGQNTN |
| MT15 | 16 | MEHNPVVMVHGIGGASENFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADPIVANSLSRLDGARAVQIHGVG |
| | | HIGLLYSSQVNSLIKEGLNGGGQNTN |
| MT16 | 17 | MEHNPVVMVHGIGGAGFNFAGIKSYLVSQGWSRDKLHA |
| | | VDFWDKTGTNYNNGPVLSRFVQKVLDETGAKKVDIVAH |
| | | SMGGANTLYYIKNLDGGNKVANVVTLGGANRLTTGKAP |
| | | PGTDPNQKILYTSIYSSADEKVSNDLSRLDGARNVQIHGV |
| | | GHIDLLYSSQVNSLIKEGLNGGGQNTN |
| MT17 | 18 | MEHNPVVMVHGIGGDSFNFAGIKSYLVSQGWSRDKLHAV |
| | | DFWDKTGTNYNNGPVLSRFVQKVLDETGAKKVDIIAHSM |
| | | GGANTLYYIKNLDGGNKVANVVTLGGANRLTTGKAPPGT |
| | | DPNQKILYTSIYSSADEKVSNDLSRLDGARNVQIHGVGHIG |
| | | LLYSSQVNSLIKEGLNGGGQNTN |
| MT18 | 19 | MEHNPVVMVHGIGGASSNFAGIKSYLVSQGWSRDKLYAV |
| | | DFWDKTGTNYNNGPVLSRFVQKVLDETGAKKVDIVAHS |
| | | MGGANTLYYIKNLDGGNKVANVVTLGGANRLTTDKALP |
| | | GTDPNQKILYTSIYSSADPIVANSLSRLDGARNVQIHGVGH |
| | | IGLLYSSQVNSLIKEGLCGGGQNTN |
| MT19 | 20 | MEHNPVVMVHGIGGASCNFAGIKSYLVSQGWSRDKLHA |
| | | VDFWDKTGTNYNNGPVLSRFVQKVLDETGAKKVDIVAH |
| | | SMGGANTLYYIKNLDGGNKVANVVTLGGANRLTTDKAL |
| | | PGTDPNQKILYTSIYSSADEKVSNDLSQLDGARNVQIHGV |
| | | EHIGLLYSSQVNSLIKEGLNGGGQNTN |
| MT20 | 21 | MEHNPVVMVHGIGGASGNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSSADPIVANSLSMLDGARNVQIHGVG |
| | | HIGLLYSPQVNSLIKEGLSGGGQNTN |
| MT21 | 22 | MEHNPVVMVHGIGGASCNFAGIKSYLVSQGWSRDKLYA |
| | | VDFWDKTGTNYNNGPVLSRFVQKVLDETGAKKVDIVAH |
| | | SMGGANTLYYIKNLDGGNKVANVVTLGGANRLTTGKAL |
| | | PGTDPNQKILYTSIYSPADMIVMNYLSQLDGARNVQIHGV |
| | | GHIGLLYSSQVNSLIKEGLNGGGQNTN |
| MT22 | 23 | MEHNPVVMVHGIGGAGFNFAGIKSYLVSQGWSRDKLHA |
| | | VDFWDMTGTNYNNGPVLSRFVQKVLDETGAKKVDIVAH |
| | | SMGGANTLYYIKNLDGGNKVANVVTLGGANRLTTGKAP |
| | | PGTDPNQKILYTSIYSSADEKVSNDLSRLDGARNVQIHGV |
| | | GHIGLLYSSQVNSLIKEGLMGGGQNTN |
| MT23 | 24 | MEHNPVVMVHGIGGAGKNFAGIKSYLVSQGWSQDKLYA |
| | | VDFWDKTGTNYNNGPVLSRFVQKVLDETGAKKVDIVAH |
| | | SMGGANTLYYIKNLDGGNKVANVVTLGGANRLTTDKAL |
| | | PGTDPNQKILYTSIYSSADRDVGNYLSRLDGARNVQIHGV |
| | | GHIDLLYSSQVNSLIKEGLTGGGQNTN |
| WT (B. | 25 | MEHNPVVMVHGIGGASFNFAGIKSYLVSQGWSRDKLYAV |
| Subtilis) | | DFWDKTGTNYNNGPVLSRFVQKVLDETGAKKVDIVAHS |
| | | MGGANTLYYIKNLDGGNKVANVVTLGGANRLTTGKALP |
| | | GTDPNQKILYTSIYSSADMIVMNYLSRLDGARNVQIHGVG |
| | | HIGLLYSSQVNSLIKEGLNGGGQNTN |

TABLE 2
Coding nucleic acid sequences corresponding to amino acid sequences
identified in final plurality of activity droplets.

| Corresponds | | |
|---|---|---|
| to mutant: | Seq. Id. No. | Nucleic Acid Sequence |
| MT1 | 26 | ATGGAACACAACCCGGTTGTAATGGTTCATGGTATTGGCGG |
| | | TGCCGGCTTCAACTTTGCGGGCATCAAATCTTATCTGGTATC |
| | | CCAGGGCTGGTCCCGTGATAAACTGCACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCCGCCGGGCACGGACCCGAACCAAAAAA |
| | | TCCTGTACACTTCCATCTACTCTTCCGCGGACGAAAAAGTTA |
| | | GCAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT2 | 27 | ATGGAACACAACCCGGTTGTAATGGTTCATGGTATTGGCGG |
| | | TGCCAGCTTCAACTTTGCGGGCATCAAATCTTATCTGGTATC |
| | | CCAGGGCTGGTCCCGTGATAAACTGTACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACCCGATTGTTG |
| | | CGAATAGCCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT3 | 28 | ATGGAACACAACCCGGTTGTAATGGTTCATGGTATTGGCGG |
| | | TGCCGGCTTCAACTTTGCGGGCATCAAATCTTATCTGGTATC |
| | | CCAGGGCTGGTCCCGTGATAAACTGTACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACCAGGATGTTA |
| | | TTAATTGCCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT4 | 29 | ATGGAACACAACCCGGTTGTAATGGTTCATGGTATTGGCGG |
| | | TGCCGGCAAAAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACCGTGATGTTG |
| | | GTAATTATCTGTCCCGTCTGGATGGCGCTCGTAATGTACAG |
| | | ATCCACGGTGTCGGCCACATCGATCTGCTGTACAGCTCCCA |
| | | GGTGAACTCTCTGATTAAAGAGGGTCTGACCGGCGGTGGCC |
| | | AGAACACTAAT |
| MT5 | 30 | ATGGAACACAACCCGGTTGTAATGGTTCATGGTATTGGCGG |
| | | TGCCAGCACCAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGATCTGCTGTACAGCTCCCA |
| | | GGTGAACTCTCTGATTAAAGAGGGTCTGAGCGGCGGTGGCC |
| | | AGAACACTAAT |
| MT6 | 31 | ATGGAACACAACCCGGTTGTAATGGTTCATGGTATTGGCGG |
| | | TGCCAGCAAAAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT7 | 32 | ATGGAACACAACCCGGTTGTAATGGTTCATGGTATTGGCGG |
| | | TGCCAGCGAAAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCAGCTGGATGGCGCTCGTAATGTACAG |
| | | ATCCACGGTGTCGGCCACATCGATCTGCTGTACAGCTCCCA |
| | | GGTGAACTCTCTGATTAAAGAGGGTCTGGATGGCGGTGGCC |
| | | AGAACACTAAT |
| MT8 | 33 | ATGGAACACAACCCGGTTGTAATGGTTCATGGTATTGGCGG |
| | | TGCCAGCGCGAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGATCTGCTGTACAGCTCCCA |
| | | GGTGAACTCTCTGATTAAAGAGGGTCTGAGCGGCGGTGGCC |
| | | AGAACACTAAT |
| MT9 | 34 | ATGGAACACAACCCGGTTGTAATGGTTCATGGTATTGGCGG |
| | | TGCCAGCTGGAACTTTGCGGGCATCAAATCTTATCTGGTAT |
| | | CCCAGGGCTGGTCCCTGGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGGTCTGCTGTACAGCCCGCA |
| | | GGTGAACTCTCTGATTAAAGAGGGTCTGCAGGGCGGTGGCC |
| | | AGAACACTAAT |
| MT10 | 35 | ATGGAACACAACCCGGTTGTAATGGTTCATGGTATTGGCGG |
| | | TGCCAGCAACAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT11 | 36 | ATGGAACACAACCCGGTTGTAATGGTTCATGGTATTGGCGG |
| | | TGCCAGCAGCAACTTTGCGGGCATCAAATCTTATCTGGTAT |
| | | CCCAGGGCTGGTCCGGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT12 | 37 | ATGGAACACAACCCGGTTGTAATGGTTCATGGTATTGGCGG |
| | | TGCCAGCAGCAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGGATGGCGGTGGCC |
| | | AGAACACTAAT |
| MT13 | 38 | ATGGAACACAACCCGGTTGTAATGGTTCATGGTATTGGCGG |
| | | TGCCAGCTTCAACTTTGCGGGCATCAAATCTTATCTGGTATC |
| | | CCAGGGCTGGTCCCGTGATAAACTGTACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACGAAGAAGTTG |
| | | ATAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT14 | 39 | ATGGAACACAACCCGGTTGTAATGGTTCATGGTATTGGCGG |
| | | TGCCAGCAAAAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACAACGATGTTG |
| | | ATAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAGCGGCGGTGGCC |
| | | AGAACACTAAT |
| MT15 | 40 | ATGGAACACAACCCGGTTGTAATGGTTCATGGTATTGGCGG |
| | | TGCCAGCGAAAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACCCGATTGTTG |
| | | CGAATAGCCTGTCCCGTCTGGATGGCGCTCGTGCGGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT16 | 41 | ATGGAACACAACCCGGTTGTAATGGTTCATGGTATTGGCGG |
| | | TGCCGGTTTCAACTTTGCGGGCATCAAATCTTATCTGGTATC |
| | | CCAGGGCTGGTCCCGTGATAAACTGCACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCCGCCGGGCACGGACCCGAACCAAAAAA |
| | | TCCTGTACACTTCCATCTACTCTTCCGCGGACGAAAAAGTTA |
| | | GCAATGATCTGTCCCGTCTGGATGGCGCTCGTAATGTACAG |
| | | ATCCACGGTGTCGGCCACATCGATCTGCTGTACAGCTCCCA |
| | | GGTGAACTCTCTGATTAAAGAGGGTCTGAACGGCGGTGGCC |
| | | AGAACACTAAT |
| MT17 | 42 | ATGGAACACAACCCGGTTGTAATGGTTCATGGTATTGGCGG |
| | | TGATAGCTTCAACTTTGCGGGCATCAAATCTTATCTGGTATC |
| | | CCAGGGCTGGTCCCGTGATAAACTGCACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCATTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCCGCCGGGCACGGACCCGAACCAAAAAA |
| | | TCCTGTACACTTCCATCTACTCTTCCGCGGACGAAAAAGTTA |
| | | GCAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT18 | 43 | ATGGAACACAACCCGGTTGTAATGGTTCATGGTATTGGCGG |
| | | TGCCAGCAGCAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGATAAAGCGCTGCCGGGCACGGACCCGAACCAAAAAA |
| | | TCCTGTACACTTCCATCTACTCTTCCGCGGACCCGATTGTTG |
| | | CGAATAGCCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGTGCGGCGGTGGCC |
| | | AGAACACTAAT |
| MT19 | 44 | ATGGAACACAACCCGGTTGTAATGGTTCATGGTATTGGCGG |
| | | TGCCAGCTGCAACTTTGCGGGCATCAAATCTTATCTGGTATC |
| | | CCAGGGCTGGTCCCGTGATAAACTGCACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGATAAAGCGCTGCCGGGCACGGACCCGAACCAAAAAA |
| | | TCCTGTACACTTCCATCTACTCTTCCGCGGACGAAAAAGTTA |
| | | GCAATGATCTGTCCCAGCTGGATGGCGCTCGTAATGTACAG |
| | | ATCCACGGTGTCGAACACATCGGTCTGCTGTACAGCTCCCA |
| | | GGTGAACTCTCTGATTAAAGAGGGTCTGAACGGCGGTGGCC |
| | | AGAACACTAAT |
| MT20 | 45 | ATGGAACACAACCCGGTTGTAATGGTTCATGGTATTGGCGG |
| | | TGCCAGCGGTAACTTTGCGGGCATCAAATCTTATCTGGTAT |
| | | CCCAGGGCTGGTCCCGTGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACCCGATTGTTG |
| | | CGAATAGCCTGTCCATGCTGGATGGCGCTCGTAATGTACAG |
| | | ATCCACGGTGTCGGCCACATCGGTCTGCTGTACAGCCCGCA |
| | | GGTGAACTCTCTGATTAAAGAGGGTCTGAGCGGCGGTGGCC |
| | | AGAACACTAAT |
| MT21 | 46 | ATGGAACACAACCCGGTTGTAATGGTTCATGGTATTGGCGG |
| | | TGCCAGCTGCAACTTTGCGGGCATCAAATCTTATCTGGTATC |
| | | CCAGGGCTGGTCCCGTGATAAACTGTACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTCCGGCGGACATGATCGTTA |
| | | TGAATTACCTGTCCCAGCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAT |
| MT22 | 47 | ATGGAACACAACCCGGTTGTAATGGTTCATGGTATTGGCGG |
| | | TGCCGGCTTCAACTTTGCGGGCATCAAATCTTATCTGGTATC |
| | | CCAGGGCTGGTCCCGTGATAAACTGCACGCAGTAGACTTCT |
| | | GGGACATGACCGGCACCAACTACAATAACGGCCCGGTTCTG |
| | | AGCCGTTTTGTCCAGAAAGTGCTGGACGAAACTGGTGCTAA |
| | | AAAGGTGGACATCGTTGCTCACAGCATGGGTGGCGCGAAC |
| | | ACTCTGTATTACATCAAAAACCTGGACGGTGGCAACAAGGT |
| | | AGCGAACGTTGTAACTCTGGGTGGCGCTAACCGTCTGACCA |
| | | CTGGTAAAGCGCCGCCGGGCACGGACCCGAACCAAAAAAT |
| | | CCTGTACACTTCCATCTACTCTTCCGCGGACGAAAAAGTTA |
| | | GCAATGATCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGATGGGCGGTGGCC |
| | | AGAACACTAAT |
| MT23 | 48 | ATGGAACACAACCCGGTTGTAATGGTTCATGGTATTGGCGG |
| | | TGCCGGCAAAAACTTTGCGGGCATCAAATCTTATCTGGTAT |
| | | CCCAGGGCTGGTCCCAGGATAAACTGTACGCAGTAGACTTC |
| | | TGGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGATAAAGCGCTGCCGGGCACGGACCCGAACCAAAAAA |
| | | TCCTGTACACTTCCATCTACTCTTCCGCGGACCGTGATGTTG |
| | | GTAATTATCTGTCCCGTCTGGATGGCGCTCGTAATGTACAG |
| | | ATCCACGGTGTCGGCCACATCGATCTGCTGTACAGCTCCCA |
| | | GGTGAACTCTCTGATTAAAGAGGGTCTGACCGGCGGTGGCC |
| | | AGAACACTAAT |
| WT (B. | 49 | ATGGAACACAACCCGGTTGTAATGGTTCATGGTATTGGCGG |
| Subtilis) | | TGCCAGCTTCAACTTTGCGGGCATCAAATCTTATCTGGTATC |
| | | CCAGGGCTGGTCCCGTGATAAACTGTACGCAGTAGACTTCT |
| | | GGGACAAGACCGGCACCAACTACAATAACGGCCCGGTTCT |
| | | GAGCCGTTTTGTCCAGAAAGTGCTGGACGAAACTGGTGCTA |
| | | AAAAGGTGGACATCGTTGCTCACAGCATGGGTGGCGCGAA |
| | | CACTCTGTATTACATCAAAAACCTGGACGGTGGCAACAAGG |
| | | TAGCGAACGTTGTAACTCTGGGTGGCGCTAACCGTCTGACC |
| | | ACTGGTAAAGCGCTGCCGGGCACGGACCCGAACCAAAAAA |
| | | TCCTGTACACTTCCATCTACTCTTCCGCGGACATGATCGTTA |
| | | TGAATTACCTGTCCCGTCTGGATGGCGCTCGTAATGTACAG |
| | | ATCCACGGTGTCGGCCACATCGGTCTGCTGTACAGCTCCCA |
| | | GGTGAACTCTCTGATTAAAGAGGGTCTGAACGGCGGTGGCC |
| | | AGAACACTAAC |

The identified thermostable mutants and WT-LipA were transformed on a pET28a+ vector backbone into BL21(DE3) competent cells (NEB C2527H) for overnight expression in liquid culture (Invitrogen, MagicMediA E. coli Expression Medium). The protein was then purified and desalted. The protein concentrations of the mutants were linearized to 0.1 mg/mL based on nanodrop measurements. A spectroscopic analysis was conducted by measuring the absorbance of the purified protein at 280 nm to determine each thermostable mutant's concentration. Each protein's purity was confirmed by running an SDS-PAGE gel.

The purified thermostable LipA mutants and the WT-LipA, were analyzed to determine their melting temperatures (Tₘ) and their heat inactivation resistance temperatures (T₅₀—the temperature at which each protein lost 50% of its activity). Eleven enzyme variants that exhibit enhanced Tₘ and T₅₀ relative to WT-LipA, as indicated in Table 3. In addition to enhanced thermostability, the present example also indicated that the thermostability of the identified enzyme mutants can be further augmented when exposed to detergent at elevated temperatures. As evidenced in FIG. 11, selected mutants from screening—specifically MT 5, 6, 7, 8, 10, and 20—exhibited a significant increase in residual enzymatic activity following a 20-minute incubation at 80° C. in the presence of detergent (Tween 80) at concentrations of 1 mg/L, 10 mg/L, and 500 mg/L. In contrast, the wild-type enzyme (WT) showed no such increase in activity when subjected to the same detergent and temperature conditions.

TABLE 3
Melting temperatures and heat inactivation resistance
temperatures of identified LipA mutants and WT-LipA.

| Mutant | Seq. | | # of | Tₘ | T₅₀ |
|---|---|---|---|---|---|
| name | Id. No. | Mutations | mutations | (C) | (C) |
| MT5 | | F17T, M134E, I135E, | 7 | 61.20 | 68 |
| | | M137D, Y139D, G158D, | | | |
| | | N174S | | | |
| MT6 | | F17K, M134E, I135E, | 5 | 59.08 | 65 |
| | | M137D, Y139D | | | |
| MT7 | | F17E, M134E, I135E, | 8 | 57.39 | 73 |
| | | M137D, Y139D, R142Q, | | | |
| | | G158D, N174D | | | |
| MT8 | | F17A, M134E, I135E, | 7 | 60.84 | 58 |
| | | M137D, Y139D, G158D, | | | |
| | | N174S | | | |
| MT9 | | F17W, R33L, M134E, | 8 | 60.14 | 67 |
| | | I135E, M137D, Y139D, | | | |
| | | S163P, N174Q | | | |
| MT10 | | F17N, M134E, I135E, | 5 | 59.66 | 55 |
| | | M137D, Y139D | | | |
| MT11 | | F17S, R33G, M134E, | 6 | 59.79 | 58 |
| | | I135E, M137D, Y139D | | | |
| MT12 | | F17S, M134E, I135E, | 6 | 58.74 | 65 |
| | | M137D, Y139D, N174D | | | |
| MT13 | | M134E, I135E, M137D, | 4 | 58.23 | 50 |
| | | Y139D | | | |
| MT14 | | F17K, M134N, I135D, | 6 | 57.58 | 52 |
| | | M137D, Y139D, N174S | | | |
| MT20 | | F17G, M134P, M137A, | 7 | 57.48 | 82 |
| | | Y139S, R142M, S163P, | | | |
| | | N174S | | | |
| WT | | | 0 | 50.57 | 48 |

These examples demonstrate the efficacy of the methods provided herein for designing and screening large libraries of nucleic acids to identify useful sequences.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used. Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present disclosure.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The contents of the electronic sequence listing (H049870773WO00-SEQ-TC.xml; Size: 61,284 bytes; and Date of Creation: Sep. 25, 2023) is herein incorporated by reference in its entirety.

What is claimed is:

1. A method, comprising:
determining one or more activity droplets of a first plurality of droplets having an activity of a target substrate, wherein at least 50% of the droplets the first plurality of droplets each comprise at least 5 distinct nucleic acid sequences;
separating nucleic acids from the one or more activity droplets of the first plurality of droplets into a second plurality of droplets; and
amplifying the nucleic acids of the one or more activity droplets.

2. A method as in claim 1, wherein the method further comprises performing steps (a)-(c) prior to performing the steps of determining the one or more activity droplets of the first plurality, separating nucleic acids from the one or more activity droplets, and amplifying the nucleic acids of the one or more activity droplets:
(a) determining one or more activity droplets of a starting plurality of droplets that contain activity of a target substrate;
(b) separating nucleic acids from the one or more activity droplets of the starting plurality of droplets into a new plurality of droplets; and
(c) amplifying the nucleic acids of the one or more activity droplets of the starting plurality of droplets.

3. A method as in claim 2, wherein the method comprises repeating steps (a)-(c) one or more times prior to performing the steps of determining the one or more activity droplets of the first plurality, separating nucleic acids from the one or more activity droplets, and amplifying the nucleic acids of the one or more activity droplets.

4. A method as in any one of the preceding claims, wherein the method comprises repeating steps (a)-(c) until no more than 50% of the droplets of the new plurality of droplets comprise no more than 10 distinct nucleic acid sequence prior to performing the steps of determining the one or more activity droplets of the first plurality, separating nucleic acids from the one or more activity droplets, and amplifying the nucleic acids of the one or more activity droplets.

5. A method, comprising:
determining one or more activity droplets of a first plurality of droplets having an activity of a target substrate, wherein the first plurality of droplets comprises greater than or equal to 10⁵ droplets containing the target substrate, and wherein at least 50% of the droplets the first plurality of droplets each comprise at least 10⁵ distinct nucleic acid sequences;
separating nucleic acids from the one or more activity droplets of the first plurality of droplets into a second plurality of droplets; and
amplifying the nucleic acids of the one or more activity droplets.

6. A method as in any one of the preceding claims, wherein the method further comprises translating proteins from the nucleic acids present within the first plurality of droplets.

7. A method, comprising:
in a first plurality of droplets comprising nucleic acids, translating proteins from the nucleic acids within the droplets, at least 50% of the droplets the first plurality of droplets containing therein at least 10⁵ distinct nucleic acid sequences, and wherein the first plurality of droplets comprises greater than or equal to 10⁵ droplets having distinct nucleic acid sequences contained therein;
determining one or more activity droplets of the first plurality of droplets that contain activity of a target substrate;
separating nucleic acids from the one or more activity droplets of the first plurality of droplets into a second plurality of droplets; and
amplifying the nucleic acids of the one or more activity droplets.

8. A method as in any one of the preceding claims, wherein the method further comprises identifying one or more nucleic acid sequences that activates the target substrate.

9. A method as in any one of the preceding claims, wherein the translating proteins is performed prior to determining the one or more droplets of the first plurality of droplets that contain activity of the target substrate.

10. A method as in any one of the preceding claims, wherein the method further comprises identifying one or more nucleic acid sequences that encodes a protein that activates the target substrate.

11. A method as in any one of claims 5-10, wherein the method further comprises steps (a)-(c):
(a) determining one or more activity droplets of a starting plurality of droplets that contain activity of a target substrate;
(b) separating nucleic acids from the one or more activity droplets of the starting plurality of droplets into a new plurality of droplets; and
(c) amplifying the nucleic acids of the one or more activity droplets of the starting plurality of droplets.

12. A method as in any one of claims 5-11, wherein the method comprises repeating steps (a)-(c) one or more times.

13. A method as in any one of claims 5-12, wherein the method comprises repeating steps (a)-(c) until no more than 50% of the droplets of the new plurality of droplets comprise no more than 1 distinct nucleic acid sequence.

14. A method as in any one of the preceding claims, wherein the first plurality of droplets comprises a nucleic acid sequence that activates the target substrate.

15. A method as in any one of the preceding claims, wherein the first plurality of droplets comprises a nucleic acid sequence that encodes a protein that activates the target substrate.

16. A method as in any one of the preceding claims, wherein the first plurality of droplets further comprises a detection agent.

17. A method as in any one of the preceding claims, wherein the first plurality of droplets comprises at least 10¹⁰ distinct nucleic acid sequences.

18. A method as in any one of the preceding claims, wherein the first plurality of droplets comprises at least 10¹¹ distinct nucleic acid sequences.

19. A method as in any one of the preceding claims, wherein the first plurality of droplets comprises at least 10¹² distinct nucleic acid sequences.

20. A method as in any one of the preceding claims, wherein a nucleic acid sequence is present in more than one droplet of the first plurality of droplets.

21. An article, comprising:
a plurality of droplets, at least 50% of the droplets containing therein at least 10⁵ distinct nucleic acid sequences and a target substrate, wherein the plurality of droplets comprises greater than or equal to 10⁵ droplets having distinct nucleic acid sequences contained therein.

22. An article as in any one of the preceding claims, wherein one or more activity droplets of the plurality of droplets contains activity of the target substrate and the target substrate within one or more of the droplets of the plurality of droplets does not contain activity of the target substrate.

23. An article as in any one of the preceding claims, wherein the plurality of droplets comprises a nucleic acid sequence that activates the target substrate.

24. An article as in any one of the preceding claims, wherein the plurality of droplets comprises a nucleic acid sequence that encodes a protein that activates the target substrate.

25. An article as in any one of the preceding claims, wherein the first plurality of droplets further comprises a detection agent.

26. An article as in any one of the preceding claims, wherein the first plurality of droplets comprises at least 10¹⁰ distinct nucleic acid sequences.

27. An article as in any one of the preceding claims, wherein the first plurality of droplets comprises at least 10¹¹ distinct nucleic acid sequences.

28. An article as in any one of the preceding claims, wherein the first plurality of droplets comprises at least 10¹² distinct nucleic acid sequences.

29. An article as in any one of the preceding claims, wherein a nucleic acid sequence is present in more than one droplet of the first plurality of droplets.

30. A method or article as in any one of the preceding claims, wherein the detection agent is configured to produce a signal in response to activity of the target substrate.

31. A method or article as in any one of the preceding claims, wherein the signal is an optical signal.

32. A method or article as in any one of the preceding claims, wherein the signal is a fluorescent signal.

33. A method or article as in any one of the preceding claims, wherein the signal is a colorimetric signal.

34. A method or article as in any one of the preceding claims, wherein the activity of the target substrate is demonstrated by reaction of the target substrate.

35. A method or article as in any one of the preceding claims, wherein the activity of the target substrate is demonstrated by binding of a protein or nucleic acid to the target substrate.

36. A method or article as in any one of the preceding claims, wherein the one or more activity droplets contain activity of the target substrate that exceeds a threshold value.

37. A method or article as in any one of the preceding claims, wherein the nucleic acid sequences are DNA sequences.

38. A method or article as in any one of the preceding claims, wherein the nucleic acid sequences are RNA sequences.

39. A method or article as in any one of the preceding claims, wherein the droplets are free of cells.

40. A composition, comprising:
an amino acid sequence at least 70% identical to one of Seq. ID. Nos. 2-24, wherein the amino acid sequence is not Seq. ID. No. 25.

41. The composition of claim 40, wherein the composition comprises one of Seq. ID. Nos. 2-24.

42. The composition of claim 40, wherein the composition is one of Seq. ID. Nos. 2-24.

43. A composition, comprising:
a nucleic acid sequence at least 70% identical to one of Seq. ID. Nos. 26-48, wherein the nucleic acid sequence is not Seq. ID. No. 49.

44. The composition of claim 43, wherein the composition comprises one of Seq. ID. Nos. 26-48.

45. The composition of claim 43, wherein the composition is one of Seq. ID. Nos. 26-48.


SEQUENCE LISTING (49 sequences)

SEQ ID NO: 1 - protein, 10 aa, synthetic construct
  SITE 1: note = 5-Carboxyfluorescein fluorophore
  SITE 10: note = 5-Carboxyfluorescein quencher
PQPQLPYPQK

SEQ ID NO: 2 - protein, 181 aa, synthetic construct
MEHNPVVMVHGIGGAGFNFAGIKSYLVSQGWSRDKLHAVDFWDKTGTNYNNGPVLSRFVQKVLDETGAKKVDIVAHSMGGANTLYYIKNLDGGNKVANVVTLGGANRLTTGKAPPGTDPNQKILYTSIYSSADEKVSNDLSRLDGARNVQIHGVGHIGLLYSSQVNSLIKEGLNGGGQNTN

SEQ ID NO: 3 - protein, 181 aa, synthetic construct
MEHNPVVMVHGIGGASFNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADPIVANSLSRLDGARNVQIHGVGHIGLLYSSQVNSLIKEGLNGGGQNTN

SEQ ID NO: 4 - protein, 181 aa, synthetic construct
MEHNPVVMVHGIGGAGFNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADQDVINCLSRLDGARNVQIHGVGHIGLLYSSQVNSLIKEGLNGGGQNTN

SEQ ID NO: 5 - protein, 181 aa, synthetic construct
MEHNPVVMVHGIGGAGKNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADRDVGNYLSRLDGARNVQIHGVGHIDLLYSSQVNSLIKEGLTGGGQNTN

SEQ ID NO: 6 - protein, 181 aa, synthetic construct
MEHNPVVMVHGIGGASTNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIDLLYSSQVNSLIKEGLSGGGQNTN

SEQ ID NO: 7 - protein, 181 aa, synthetic construct
MEHNPVVMVHGIGGASKNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIGLLYSSQVNSLIKEGLNGGGQNTN

SEQ ID NO: 8 - protein, 181 aa, synthetic construct
MEHNPVVMVHGIGGASENFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSQLDGARNVQIHGVGHIDLLYSSQVNSLIKEGLDGGGQNTN

SEQ ID NO: 9 - protein, 181 aa, synthetic construct
MEHNPVVMVHGIGGASANFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIDLLYSSQVNSLIKEGLSGGGQNTN

SEQ ID NO: 10 - protein, 181 aa, synthetic construct
MEHNPVVMVHGIGGASWNFAGIKSYLVSQGWSLDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIGLLYSPQVNSLIKEGLQGGGQNTN

SEQ ID NO: 11 - protein, 181 aa, synthetic construct
MEHNPVVMVHGIGGASNNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIGLLYSSQVNSLIKEGLNGGGQNTN

SEQ ID NO: 12 - protein, 181 aa, synthetic construct
MEHNPVVMVHGIGGASSNFAGIKSYLVSQGWSGDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIGLLYSSQVNSLIKEGLNGGGQNTN

SEQ ID NO: 13 - protein, 181 aa, synthetic construct
MEHNPVVMVHGIGGASSNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIGLLYSSQVNSLIKEGLDGGGQNTN

SEQ ID NO: 14 - protein, 181 aa, synthetic construct
MEHNPVVMVHGIGGASFNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADEEVDNDLSRLDGARNVQIHGVGHIGLLYSSQVNSLIKEGLNGGGQNTN

SEQ ID NO: 15 - protein, 181 aa, synthetic construct
MEHNPVVMVHGIGGASKNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADNDVDNDLSRLDGARNVQIHGVGHIGLLYSSQVNSLIKEGLSGGGQNTN

SEQ ID NO: 16 - protein, 181 aa, synthetic construct
MEHNPVVMVHGIGGASENFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADPIVANSLSRLDGARAVQIHGVGHIGLLYSSQVNSLIKEGLNGGGQNTN

SEQ ID NO: 17 - protein, 181 aa, synthetic construct
MEHNPVVMVHGIGGAGFNFAGIKSYLVSQGWSRDKLHAVDFWDKTGTNYNNGPVLSRFVQKVLDETGAKKVDIVAHSMGGANTLYYIKNLDGGNKVANVVTLGGANRLTTGKAPPGTDPNQKILYTSIYSSADEKVSNDLSRLDGARNVQIHGVGHIDLLYSSQVNSLIKEGLNGGGQNTN

SEQ ID NO: 18 - protein, 181 aa, synthetic construct
MEHNPVVMVHGIGGDSFNFAGIKSYLVSQGWSRDKLHAVDFWDKTGTNYNNGPVLSRFVQKVLDETGAKKVDIIAHSMGGANTLYYIKNLDGGNKVANVVTLGGANRLTTGKAPPGTDPNQKILYTSIYSSADEKVSNDLSRLDGARNVQIHGVGHIGLLYSSQVNSLIKEGLNGGGQNTN

SEQ ID NO: 19 - protein, 181 aa, synthetic construct
MEHNPVVMVHGIGGASSNFAGIKSYLVSQGWSRDKLYAVDFWDKTGTNYNNGPVLSRFVQKVLDETGAKKVDIVAHSMGGANTLYYIKNLDGGNKVANVVTLGGANRLTTDKALPGTDPNQKILYTSIYSSADPIVANSLSRLDGARNVQIHGVGHIGLLYSSQVNSLIKEGLCGGGQNTN

SEQ ID NO: 20 - protein, 181 aa, synthetic construct
MEHNPVVMVHGIGGASCNFAGIKSYLVSQGWSRDKLHAVDFWDKTGTNYNNGPVLSRFVQKVLDETGAKKVDIVAHSMGGANTLYYIKNLDGGNKVANVVTLGGANRLTTDKALPGTDPNQKILYTSIYSSADEKVSNDLSQLDGARNVQIHGVEHIGLLYSSQVNSLIKEGLNGGGQNTN

SEQ ID NO: 21 - protein, 181 aa, synthetic construct
MEHNPVVMVHGIGGASGNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADPIVANSLSMLDGARNVQIHGVGHIGLLYSPQVNSLIKEGLSGGGQNTN

SEQ ID NO: 22 - protein, 181 aa, synthetic construct
MEHNPVVMVHGIGGASCNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSPADMIVMNYLSQLDGARNVQIHGVGHIGLLYSSQVNSLIKEGLNGGGQNTN

SEQ ID NO: 23 - protein, 181 aa, synthetic construct
MEHNPVVMVHGIGGAGFNFAGIKSYLVSQGWSRDKLHAVDFWDMTGTNYNNGPVLSRFVQKVLDETGAKKVDIVAHSMGGANTLYYIKNLDGGNKVANVVTLGGANRLTTGKAPPGTDPNQKILYTSIYSSADEKVSNDLSRLDGARNVQIHGVGHIGLLYSSQVNSLIKEGLMGGGQNTN

SEQ ID NO: 24 - protein, 181 aa, synthetic construct
MEHNPVVMVHGIGGAGKNFAGIKSYLVSQGWSQDKLYAVDFWDKTGTNYNNGPVLSRFVQKVLDETGAKKVDIVAHSMGGANTLYYIKNLDGGNKVANVVTLGGANRLTTDKALPGTDPNQKILYTSIYSSADRDVGNYLSRLDGARNVQIHGVGHIDLLYSSQVNSLIKEGLTGGGQNTN

SEQ ID NO: 25 - protein, 181 aa, Bacillus subtilis
MEHNPVVMVHGIGGASFNFAGIKSYLVSQGWSRDKLYAVDFWDKTGTNYNNGPVLSRFVQKVLDETGAKKVDIVAHSMGGANTLYYIKNLDGGNKVANVVTLGGANRLTTGKALPGTDPNQKILYTSIYSSADMIVMNYLSRLDGARNVQIHGVGHIGLLYSSQVNSLIKEGLNGGGQNTN

SEQ ID NO: 26 - DNA, 543 bp, synthetic construct
  source 1..543: mol_type = other DNA
atggaacacaacccggttgtaatggttcatggtattggcggtgccggcttcaactttgcgggcatcaaatcttatctggtatcccagggctggtcccgtgataaactgcacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgccgccgggcacggacccgaaccaaaaaatcctgtacacttccatctactcttccgcggacgaaaaagttagcaatgatctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 27 - DNA, 543 bp, synthetic construct
  source 1..543: mol_type = other DNA
atggaacacaacccggttgtaatggttcatggtattggcggtgccagcttcaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacccgattgttgcgaatagcctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 28 - DNA, 543 bp, synthetic construct
  source 1..543: mol_type = other DNA
atggaacacaacccggttgtaatggttcatggtattggcggtgccggcttcaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggaccaggatgttattaattgcctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 29 - DNA, 543 bp, synthetic construct
  source 1..543: mol_type = other DNA
atggaacacaacccggttgtaatggttcatggtattggcggtgccggcaaaaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggaccgtgatgttggtaattatctgtcccgtctggatggcgctcgtaatgtacagatccacggtgtcggccacatcgatctgctgtacagctcccaggtgaactctctgattaaagagggtctgaccggcggtggccagaacactaat

SEQ ID NO: 30 - DNA, 543 bp, synthetic construct
  source 1..543: mol_type = other DNA
atggaacacaacccggttgtaatggttcatggtattggcggtgccagcaccaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcgatctgctgtacagctcccaggtgaactctctgattaaagagggtctgagcggcggtggccagaacactaat

SEQ ID NO: 31 - DNA, 543 bp, synthetic construct
  source 1..543: mol_type = other DNA
atggaacacaacccggttgtaatggttcatggtattggcggtgccagcaaaaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 32 - DNA, 543 bp, synthetic construct
  source 1..543: mol_type = other DNA
atggaacacaacccggttgtaatggttcatggtattggcggtgccagcgaaaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccagctggatggcgctcgtaatgtacagatccacggtgtcggccacatcgatctgctgtacagctcccaggtgaactctctgattaaagagggtctggatggcggtggccagaacactaat

SEQ ID NO: 33 - DNA, 543 bp, synthetic construct
  source 1..543: mol_type = other DNA
atggaacacaacccggttgtaatggttcatggtattggcggtgccagcgcgaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcgatctgctgtacagctcccaggtgaactctctgattaaagagggtctgagcggcggtggccagaacactaat

SEQ ID NO: 34 - DNA, 543 bp, synthetic construct
  source 1..543: mol_type = other DNA
atggaacacaacccggttgtaatggttcatggtattggcggtgccagctggaactttgcgggcatcaaatcttatctggtatcccagggctggtccctggataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcggtctgctgtacagcccgcaggtgaactctctgattaaagagggtctgcagggcggtggccagaacactaat

SEQ ID NO: 35 - DNA, 543 bp, synthetic construct
  source 1..543: mol_type = other DNA
atggaacacaacccggttgtaatggttcatggtattggcggtgccagcaacaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 36 - DNA, 543 bp, synthetic construct
  source 1..543: mol_type = other DNA
atggaacacaacccggttgtaatggttcatggtattggcggtgccagcagcaactttgcgggcatcaaatcttatctggtatcccagggctggtccggtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 37 - DNA, 543 bp, synthetic construct
  source 1..543: mol_type = other DNA
atggaacacaacccggttgtaatggttcatggtattggcggtgccagcagcaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcggtctgctgtacagctcccaggtgaactctctgattaaagagggtctggatggcggtggccagaacactaat

SEQ ID NO: 38 - DNA, 543 bp, synthetic construct
  source 1..543: mol_type = other DNA
atggaacacaacccggttgtaatggttcatggtattggcggtgccagcttcaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacgaagaagttgataatgatctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 39 - DNA, 543 bp, synthetic construct
  source 1..543: mol_type = other DNA
atggaacacaacccggttgtaatggttcatggtattggcggtgccagcaaaaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacaacgatgttgataatgatctgtcccgtctggatggcgctcgtaatgtacagatccacggtgtcggccacatcggtctgctgtacagctcccaggtgaactctctgattaaagagggtctgagcggcggtggccagaacactaat

SEQ ID NO: 40 - DNA, 543 bp, synthetic construct
  source 1..543: mol_type = other DNA
atggaacacaacccggttgtaatggttcatggtattggcggtgccagcgaaaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacccgattgttgcgaatagcctgtcccgtctggatggcgctcgtgcggtacagatccacggtgtcggccacatcggtctgctgtacagctcccaggtgaactctctgattaaagagggtctgaacggcggtggccagaacactaat

SEQ ID NO: 41 - DNA, 543 bp, synthetic construct
  source 1..543: mol_type = other DNA
atggaacacaacccggttgtaatggttcatggtattggcggtgccggtttcaactttgcgggcatcaaatcttatctggtatcccagggctggtcccgtgataaactgcacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgccgccgggcacggacccgaaccaaaaaatcctgtacacttccatctactcttccgcggacgaaaaagttagcaatgatctgtcccgtctggatggcgctcgtaatgtacagatccacggtgtcggccacatcgatctgctgtacagctcccaggtgaactctctgattaaagagggtctgaacggcggtggccagaacactaat

SEQ ID NO: 42 - DNA, 543 bp, synthetic construct
  source 1..543: mol_type = other DNA
atggaacacaacccggttgtaatggttcatggtattggcggtgatagcttcaactttgcgggcatcaaatcttatctggtatcccagggctggtcccgtgataaactgcacgcagtagacttctgggacaagaccggcaccaactacaataacggcccggttctgagccgttttgtccagaaagtgctggacgaaactggtgctaaaaaggtggacatcattgctcacagcatgggtggcgcgaacactctgtattacatcaaaaacctggacggtggcaacaaggtagcgaacgttgtaactctgggtggcgctaaccgtctgaccactggtaaagcgccgccgggcacggacccgaaccaaaaaatcctgtacacttccatctactcttccgcggacgaaaaagttagcaatgatctgtcccgtctggatggcgctcgtaatgtacagatccacggtgtcggccacatcggtctgctgtacagctcccaggtgaactctctgattaaagagggtctgaacggcggtggccagaacactaat

SEQ ID NO: 43 - DNA, 543 bp, synthetic construct
  source 1..543: mol_type = other DNA
atggaacacaacccggttgtaatggttcatggtattggcggtgccagcagcaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactgataaagcgctgccgggcacggacccgaaccaaaaaatcctgtacacttccatctactcttccgcggacccgattgttgcgaatagcctgtcccgtctggatggcgctcgtaatgtacagatccacggtgtcggccacatcggtctgctgtacagctcccaggtgaactctctgattaaagagggtctgtgcggcggtggccagaacactaat

SEQ ID NO: 44 - DNA, 543 bp, synthetic construct
  source 1..543: mol_type = other DNA
atggaacacaacccggttgtaatggttcatggtattggcggtgccagctgcaactttgcgggcatcaaatcttatctggtatcccagggctggtcccgtgataaactgcacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactgataaagcgctgccgggcacggacccgaaccaaaaaatcctgtacacttccatctactcttccgcggacgaaaaagttagcaatgatctgtcccagctggatggcgctcgtaatgtacagatccacggtgtcgaacacatcggtctgctgtacagctcccaggtgaactctctgattaaagagggtctgaacggcggtggccagaacactaat

SEQ ID NO: 45 - DNA, 543 bp, synthetic construct
  source 1..543: mol_type = other DNA
atggaacacaacccggttgtaatggttcatggtattggcggtgccagcggtaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacccgattgttgcgaatagcctgtccatgctggatggcgctcgtaatgtacagatccacggtgtcggccacatcggtctgctgtacagcccgcaggtgaactctctgattaaagagggtctgagcggcggtggccagaacactaat

SEQ ID NO: 46 - DNA, 543 bp, synthetic construct
  source 1..543: mol_type = other DNA
atggaacacaacccggttgtaatggttcatggtattggcggtgccagctgcaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactctccggcggacatgatcgttatgaattacctgtcccagctggatggcgctcgtaatgtacagatccacggtgtcggccacatcggtctgctgtacagctcccaggtgaactctctgattaaagagggtctgaacggcggtggccagaacactaat

SEQ ID NO: 47 - DNA, 543 bp, synthetic construct
  source 1..543: mol_type = other DNA
atggaacacaacccggttgtaatggttcatggtattggcggtgccggcttcaactttgcgggcatcaaatcttatctggtatcccagggctggtcccgtgataaactgcacgcagtagacttctgggacatgaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgccgccgggcacggacccgaaccaaaaaatcctgtacacttccatctactcttccgcggacgaaaaagttagcaatgatctgtcccgtctggatggcgctcgtaatgtacagatccacggtgtcggccacatcggtctgctgtacagctcccaggtgaactctctgattaaagagggtctgatgggcggtggccagaacactaat

SEQ ID NO: 48 - DNA, 543 bp, synthetic construct
  source 1..543: mol_type = other DNA
atggaacacaacccggttgtaatggttcatggtattggcggtgccggcaaaaactttgcgggcatcaaatcttatctggtatcccagggctggtcccaggataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactgataaagcgctgccgggcacggacccgaaccaaaaaatcctgtacacttccatctactcttccgcggaccgtgatgttggtaattatctgtcccgtctggatggcgctcgtaatgtacagatccacggtgtcggccacatcgatctgctgtacagctcccaggtgaactctctgattaaagagggtctgaccggcggtggccagaacactaat

SEQ ID NO: 49 - DNA, 543 bp, Bacillus subtilis
  source 1..543: mol_type = genomic DNA
atggaacacaacccggttgtaatggttcatggtattggcggtgccagcttcaactttgcgggcatcaaatcttatctggtatcccagggctggtcccgtgataaactgtacgcagtagacttctgggacaagaccggcaccaactacaataacggcccggttctgagccgttttgtccagaaagtgctggacgaaactggtgctaaaaaggtggacatcgttgctcacagcatgggtggcgcgaacactctgtattacatcaaaaacctggacggtggcaacaaggtagcgaacgttgtaactctgggtggcgctaaccgtctgaccactggtaaagcgctgccgggcacggacccgaaccaaaaaatcctgtacacttccatctactcttccgcggacatgatcgttatgaattacctgtcccgtctggatggcgctcgtaatgtacagatccacggtgtcggccacatcggtctgctgtacagctcccaggtgaactctctgattaaagagggtctgaacggcggtggccagaacactaac